SEQUENCE LISTING (27 sequences)

SEQ ID NO: 1 - DNA, 17 bp, Artificial
  Synthetic DNA
taatacgactcactata

SEQ ID NO: 2 - DNA, 17 bp, Artificial
  Synthetic DNA
atttaggtgacactata

SEQ ID NO: 3 - DNA, 17 bp, Artificial
  Synthetic DNA
aattaaccctcactaaa

SEQ ID NO: 4 - DNA, 60 bp, Artificial
  Synthetic DNA
tgcagggcggaccgatcacatgaagcagcacgacttcattgcctatagtgagtcgtatta

SEQ ID NO: 5 - DNA, 19 bp, Artificial
  Synthetic DNA
taatacgactcactatagg

SEQ ID NO: 6 - DNA, 69 bp, Artificial
  Synthetic DNA
gacttgtgcagggcggactatgaagagatacgccctgcattgccctctccctatagtgagtcgtattag

SEQ ID NO: 7 - DNA, 19 bp, Artificial
  Synthetic DNA
taatacgactcactatagg

SEQ ID NO: 8 - DNA, 17 bp, Artificial
  Synthetic DNA
gacttgtgcagggcgga

SEQ ID NO: 9 - DNA, 14 bp, Artificial
  Synthetic DNA
gggagagggcaatg

SEQ ID NO: 10 - DNA, 28 bp, Artificial
  Synthetic DNA
aatgatacggcgaccaccgannnnnnnn

SEQ ID NO: 11 - DNA, 21 bp, Artificial
  Synthetic DNA
tcgtatgccgtcttctgcttg

SEQ ID NO: 12 - DNA, 23 bp, Artificial
  Synthetic DNA
caagcagaagacggcatacgann

SEQ ID NO: 13 - DNA, 21 bp, Artificial
  Synthetic DNA
gaacaattgcttttacagatg

SEQ ID NO: 14 - DNA, 20 bp, Artificial
  Synthetic DNA
cattaaaaccgggaggtaga

SEQ ID NO: 15 - DNA, 62 bp, Artificial
  Synthetic DNA
cgtactagtatggagcagaagctgatctcagaggaggacctgatggatgtattcatgaaagg

SEQ ID NO: 16 - DNA, 17 bp, Artificial
  Synthetic DNA
tcttaggcttcaggttc

SEQ ID NO: 17 - DNA, 21 bp, Artificial
  Synthetic DNA
tcgtatgccgtcttctgcttg

SEQ ID NO: 18 - DNA, 20 bp, Artificial
  Synthetic DNA
cattaaaaccgggaggtaga

SEQ ID NO: 19 - DNA, 28 bp, Artificial
  Synthetic DNA
aggttctagagctcgaagcggccgctct

SEQ ID NO: 20 - DNA, 21 bp, Artificial
  Synthetic DNA
gaacaattgcttttacagatg

SEQ ID NO: 21 - DNA, 20 bp, Artificial
  Synthetic DNA
aatgatacggcgaccaccga

SEQ ID NO: 22 - DNA, 44 bp, Artificial
  Synthetic DNA
ttgatgatgcatctgagcatctgatgtttaaactcatgctgaag

SEQ ID NO: 23 - DNA, 32 bp, Artificial
  Synthetic DNA
taatacgactcactatagggagataggcaatg

SEQ ID NO: 24 - DNA, 39 bp, Artificial
  Synthetic DNA
gtgcattgaattcgaccactaaaggaactcatgctgaag

SEQ ID NO: 25 - DNA, 23 bp, Artificial
  Synthetic DNA
tgcgaattcgggagagggcaatg

SEQ ID NO: 26 - DNA, 20 bp, Artificial
  Synthetic DNA
aatgatacggcgaccaccga

SEQ ID NO: 27 - DNA, 21 bp, Artificial
  Synthetic DNA
caagcagaagacggcatacga

(19) United States
(12) Patent Application Publication
(10) Pub. No.: US 2011/0172105 A1
(43) Pub. Date: Jul. 14, 2011

(54) GREPSEQ: An Almost Inexhaustible, Cost-Effective, High-Throughput Protocol for the Generation of Selector Sequences

(75) Inventors: Fred H. Gage, La Jolla, CA (US); Jonathan Scolnick, San Diego (CA); Gene Wei Ming Yeo, La Jolla, CA (US)

(73) Assignee: Salk Institute for Biological Studies, La Jolla, CA (US)

(21) Appl. No.: 12/996,008

(22) PCT Filed: Jun. 4, 2009
(86) PCT No.: PCT/US2009/003417

§ 371 Date: Mar. 30, 2011

Related U.S. Application Data
(60) Provisional application No. 61/131,004, filed on Jun. 4, 2008.

(51) Int. Cl. C40B 20/00; C07H 21/00; C40B 40/06; C12P 19/34

(52) U.S. Cl. 506/2; 536/23.1; 506/16; 435/91.5

(57) ABSTRACT

Provided are compositions, libraries, and methods for the synthesis of transcripts that can be processed to produce nucleic acid capture probes. Also provided methods for using such nucleic acid capture probes in a variety of downstream applications, including, e.g., determining the sequence of an exon-exon junction.

### CROSS-REFERENCE TO RELATED APPLICATIONS

This application claims priority to and benefit of U.S. Provisional Patent Application 61/131,004, entitled, “GREPSEQ: An Almost Inexhaustible, Cost-Effective, High-Throughput Protocol for the Generation of Selector Sequences,” by Yeo, Scolnick, and Gage, filed Jun. 4, 2008, the disclosure of which is incorporated herein in its entirety for all purposes.

### FIELD OF THE INVENTION

The present invention relates to genome-wide nucleic acid interrogation techniques. Specifically, the invention provides compositions, systems, kits, and methods for the production of reagents that can be used to enrich sequences of interest from a sample comprising a population of nucleic acids.

### BACKGROUND OF THE INVENTION

Nucleic acid sequence data is valuable in myriad applications in biological research and molecular medicine, including determining the hereditary factors in disease, in developing new methods to detect disease and to guide therapy (van de Vijver et al. (2002) “A gene-expression signature as a predictor of survival in breast cancer,” New England Journal of Medicine 347: 1999-2009), in drug development, and in providing a rational basis for personalized medicine. Because reference genome sequences for many organisms are now publicly available, cataloging sequence variations and understanding their biological consequences has become a major research goal.

The complete genomes of two individuals have recently been sequenced. One genome was sequenced using Sanger dideoxy technology (Levy et al. (2007) “The Diploid Genome Sequence of an Individual Human.” PLoS Biol 5: e254) at a cost of $10,000,000, and the other was sequenced using a high-throughput sequencing system available from 454 Life Sciences (Wheeler et al. (2008) “The complete genome of an individual by massively parallel DNA sequencing.” Nature 452: 872-876) at a cost of $2,000,000. Though the costs of sequencing the second human genome were reduced by a factor of 5 relative to the cost of sequencing the first, using even recently developed high-throughput sequencing technologies can be too costly and laborious to sequence the complete genomes of more than a small number of individuals. A more cost-effective alternative to whole genome sequencing is targeted resequencing, e.g., the sequencing of one or more gene segments, regions, or genomic loci of interest of nucleic acid samples of interest. Resequencing can be used to identify, e.g., genotype, known mutations in a nucleic acid of interest and/or to perform variation analysis, e.g., scan a nucleic of interest for any mutation in a given target region. The targeted resequencing of candidate genes or other genomic regions can be a particularly useful method of detecting mutations associated with various complex human diseases, including cancer, heart disease, and others.

Targeted resequencing can be integrated with any of a variety of high-throughput DNA sequencing systems (reviewed in, e.g., Chan et al. (2005) “Advances in Sequencing Technology” Mutation Research 573: 13-40) to permit large-scale resequencing efforts. Many commercial high-throughput sequencing systems rely on multiplexed direct sequencing methods, e.g., “sequencing by synthesis” (SBS), in which each base position in a DNA chain is determined individually. See, e.g., Eid et al. (2008) “Real-Time DNA Sequencing from Single Polymerase Molecules.” Science 323: 133-138; Levene et al. (2003) “Zero Mode Waveguides for Single Molecule Analysis at High Concentrations,” Science 299: 682-686; Mercier et al. (2005) “Solid Phase DNA Amplification: A Brownian Dynamics Study of Crowding Effects.” Biophysical Journal 89: 32-42; Nyren (2007) “The History of Pyrosequencing.” Methods Mol Biol 373: 1-14; Bennett et al. (2005) “Toward the 1,000 dollars human genome.” Pharmacogenomics 6: 373-382; Bennett S. (2004) “Solexa Ltd.” Pharmacogenomics 5: 433-438; and Bentley (2006) “Whole genome re-sequencing.” Curr Opin Genet Dev 16: 545-52. Other commercial sequencing systems rely on indirect methods of determining a sequence, e.g., sequencing by hybridization (SBH), in which a sequence of a DNA is assembled based upon data obtained from hybridization experiments performed to determine the oligonucleotide content of the DNA chain. See, e.g., Drmanac et al. (2002) “Sequencing by hybridization (SBH): advantages, achievements, and opportunities.” Adv Biochem Eng Biotechnol 77: 75-101.

However, one of the major challenges of resequencing is the efficient isolation of the target nucleic acids to be sequenced. Typically, PCR has been used to amplify regions of interest from, e.g., a nucleic acid or population of nucleic acids extracted from a biological sample in preparation for resequencing. However, using PCR to amplify regions of interest in, e.g., a genome, a population of cDNAs, or a population of RNAs, for resequencing, can limit the length of the sequence that is amplified. Repetitive regions, which are typical of complex genomes, can be difficult to amplify using PCR. Furthermore, multiplexing PCR for the enrichment of, e.g., several thousand regions of interest in a nucleic acid sample, can be both expensive and labor-intensive.

High-density DNA microarray technologies that permit the high-throughput, multiplex detection of nucleotide sequence variation have been developed for SBH (Sapolsky et al. (1999) “High-throughput polymorphism screening and genotyping with high-density oligonucleotide arrays” Genetic Analysis: Biomolecular Engineering 14: 187-192; Dong et al. (2001) “Flexible Use of High-Density Oligonucleotide Arrays for Single-Nucleotide Polymorphism Discovery and Validation.” Genome 11: 1418-1424). In one approach that uses such arrays, nucleic acid fragments, e.g., from sheared genomic DNA, a population of cDNAs, or from another kind of nucleic acid, are labeled and hybridized to a variation detection array (VDA). Sequence variations are manifested as changes in hybridization intensity to individual oligonucleotide probes in the array. However, the accuracy of the results obtained via SBH techniques depends on the careful design of the probes included on the array, and cost considerations can significantly restrict array modifications and reformatting for optimization. The identity of the sequence changes identified using VDAs can often be established only after subsequent sequencing of the recovered genomic fragments. Furthermore, deletion and frameshift mutations can be difficult to detect using VDAs because genomic fragments comprising such mutation can fail to hybridize altogether to the probes on an array.

What is needed in the art are methods and compositions that produce reagents that permit rapid, accurate, and economical enrichment of, e.g., up to thousands of selected regions of interest from a nucleic acid sample, e.g., in preparation for resequencing. Methods and compositions that can ideally be applied to a variety of genomic applications, including mutation analysis, gene expression analysis, and/or identifying and characterizing novel RNA isoforms would be useful. In addition, such methods and systems are most beneficially automatable and/or compatible with current and future high-throughput sequencing systems. The invention described herein fulfills these and other needs, as will be apparent upon review of the following.

### SUMMARY OF THE INVENTION

The present invention is generally directed to compositions and methods for transcribing RNAs from nucleic acid(s) that are tethered to a solid support. The methods and compositions of the invention are beneficially used to produce nucleic acid capture probes, e.g., probes that permit the targeted enrichment of nucleic acids comprising specific sequences of interest from a nucleic acid sample of interest. The methods provided herein can beneficially minimize the costs of targeted resequencing and can reduce the need for labor-intensive large-scale PCR. Such probes can be used for a variety of genomic interrogation applications, including, but not limited to, e.g., identifying exon-exon junctions in target nucleic acids, identifying alternate transcription start sites in target nucleic acids, identifying alternate 3′UTR/polyA sites in target nucleic acids, and others. The probes can also be used to detect mutations and polymorphisms in target subsequences of interest. The methods and compositions provided herein can advantageously be used in combination with high-throughput sequencing systems, and systems for the high-throughput transcription, reverse transcription, and/or copying of nucleic acids.

Accordingly, in one aspect, the invention provides compositions that comprise a solid support and at least one nucleic acid. In the compositions, the 5′ end of the nucleic acid is tethered to the solid support, and the 3′ end region of the nucleic acid comprises at least one strand of a promoter sequence, e.g., a T4 promoter sequence, a T7 promoter sequence, a T3 promoter sequence, or an SP6 promoter sequence, that is recognized by an RNA polymerase, e.g., a T4 RNA polymerase, T7 RNA polymerase, a T3 RNA polymerase, or an SP6 RNA polymerase. The nucleic acid of the compositions is capable of being transcribed by an RNA polymerase from the promoter towards the 5′ end when the promoter sequence is sufficiently double stranded for recognition by an RNA polymerase, e.g., including, but not limited to, any one of the RNA polymerases described above.

The nucleic acid tethered to the solid support can optionally comprise a selector subsequence, e.g., a sequence that can hybridize to, e.g., an exon, an intron, an exon-exon boundary, a 3′ UTR/polyA site, a transcription start site, an shRNA sequence, or a subsequence of an miRNA, downstream of the promoter sequence that can be transcribed by the RNA polymerase. The nucleic acid can optionally comprise a constant region downstream of the selector subsequence, and the constant region can optionally comprise or encode at least one strand of a unique restriction endonuclease recognition site. The compositions can optionally comprise a primer that can hybridize to the promoter sequence and permit the RNA polymerase, e.g., a T4 RNA polymerase, T7 RNA polymerase, a T3 RNA polymerase, or an SP6 RNA polymerase, to transcribe the nucleic acid downstream of the promoter.

The solid support of the compositions can optionally comprise a polymer, a ceramic, glass, a metal, a metalloid, or a magnetic material. Optionally, the solid support can comprise a planar substrate, a bead, a slide, a microscope slide, or a micro-well plate. In certain embodiments, the compositions provided by the invention can optionally comprise an array of nucleic acids, e.g., a plurality of copies of each of a plurality of nucleic acid sequence types, tethered to a solid support. The nucleic acid sequence types in the array can each optionally comprise a plurality of selector subsequences, e.g., an exon, an intron, an exon-exon boundary, a 3′UTR/polyA site, a transcription start site, an shRNA sequence, or a subsequence of an miRNA.

The compositions provided by the invention can optionally be included in systems that comprise a production module that produces transcripts of the nucleic acid. In addition to the production module, the systems can optionally include a processing module that copies or transcribes the transcript and a sequencing module that sequences products of the processing module.

In a related aspect, the invention provides methods of producing an RNA. The methods include providing a solid support to which at least one nucleic acid has been tethered, e.g., enzymatically or chemically coupled, by its 5′ end. The tethered nucleic acid comprises or encodes at least one strand of a promoter sequence recognized by an RNA polymerase at its 3′ end region. The methods include annealing a primer to the promoter sequence to provide the promoter recognized by the RNA polymerase and transcribing the nucleic acid with the RNA polymerase. In these methods, the polymerase travels along the nucleic acid toward the 5′ end during transcription, thereby producing the RNA. The methods of producing an RNA can optionally include producing a cDNA from the RNA. Optionally, the methods can further include sequencing at least a portion of the cDNA or a complementary sequence thereof.

The invention also provides related methods of synthesizing a tagged single-stranded nucleic acid capture probe. In general, the methods include providing a solid support to which at least one nucleic acid has been tethered by its 5′end. The tethered nucleic acid includes a selector subsequence of interest and at least one strand of a promoter sequence recognized by an RNA polymerase upstream of the selector subsequence. Optionally, the promoter is double-stranded wherein the promoter comprises a primer annealed to the nucleic acid. The methods include transcribing the nucleic acid with the RNA polymerase to produce an RNA, reverse transcribing the RNA with a reverse transcriptase to produce a tagged single-stranded cDNA, and removing at least one nucleotide from the 3′ end of the tagged single-stranded cDNA, e.g., with an enzyme that has a 3′-5′ exonuclease activity, to produce the nucleic acid capture probe.

A primer can optionally be annealed to the nucleic acid to produce a double-stranded promoter from which an RNA can be transcribed. Reverse transcribing an RNA to produce a single-stranded tagged cDNA using these methods can optionally include annealing a primer comprising a 5′ tagged end to the 3′ end of an RNA and extending the primer with a reverse transcriptase to form an RNA:DNA duplex that comprises a cDNA strand with a tagged 5′ end, and separating the strands of each duplex from one another, e.g., by denaturing the RNA:DNA duplex or by digesting the RNA strand of the RNA:DNA duplex with RNAse H.

A primer comprising 5′ a tagged end can be annealed to the 3′ end of an RNA using any of a variety of approaches. For example, the tagged primer can optionally comprise a sequence complementary to the sequence at the 3′ end of the RNA. Optionally, a polyA tail can be added to the 3′ end of the RNA, e.g., via enzymatic addition of adenosine residues by a polyA polymerase, a terminal transferase, or an RNA ligase, and a 5′-tagged polyT primer can be annealed to the polyA tail. The tag at the 5′ end of the primer that is annealed to the 3′ end of the RNA or to the polyA tail can optionally include one or more ligand, blocking group, phosphorylated nucleotide, phosphorothioated nucleotide, biotinylated nucleotide, digoxigenin-labeled nucleotide, methylated nucleotide, uracil, sequence capable of forming a hairpin structure, oligonucleotide hybridization site, restriction endonuclease recognition site, promoter sequence, sample or library identification sequence, and/or cis regulatory sequence.

The invention also provides a variation of the methods described above. In this second set of methods of synthesizing a single-stranded nucleic acid capture probe, an RNA is synthesized from a nucleic acid tethered to a solid support, as described previously. The second set of methods includes reverse transcribing the RNA with a reverse transcriptase to produce a double-stranded cDNA with one tagged end, removing at least one nucleotide base pair from an untagged end of the double-stranded cDNA, and separating the strands of the double-stranded cDNA from one another.

Reverse transcribing the RNAs can optionally include annealing a tagged primer to a 3′ end of the RNA, extending the tagged primer with a reverse transcriptase to form a double-stranded RNA-DNA duplex comprising a cDNA strand with a tagged 5′ end, separating the strands of the RNA:DNA duplex, annealing an untagged primer to the 3′ end of the cDNA strand, and extending the untagged primer with a DNA polymerase to produce the double-stranded cDNA that comprises one tagged strand. The DNA polymerase used to extend the untagged primer can optionally be an E. coli DNA polymerase I, a Taq polymerase, a T7 DNA polymerase, a T3 DNA polymerase, a phi29 DNA polymerase, a Vent DNA polymerase, a Pfu DNA polymerase, a Bst DNA polymerase, and a 9°Nm™ DNA polymerase.

Removing at least one nucleotide base pair from an untagged end of the double-stranded cDNA that comprises one tagged strand can optionally comprise digesting the double-stranded cDNA with an endonuclease at a site proximal to the untagged end of the double-stranded cDNA such that the nucleotide base pair is removed from the double-stranded cDNA. Separating the strands of the double-stranded cDNA that comprises one tagged strand can optionally include denaturing the double-stranded cDNA or digesting the untagged strand with a lambda nuclease.

Any of the methods for synthesizing a single-stranded nucleic acid capture probe can optionally include sequencing at least a portion of the tagged single-stranded capture nucleic acid, or a complementary sequence thereof.

In a related aspect, the invention also provides nucleic acid libraries that include one or more arrays. Each array comprises a solid support and a plurality of nucleic acids, each of which is tethered to the solid support at one end, e.g., a 5′end. Each of the nucleic acids in the plurality also comprises a strand of an RNA polymerase promoter and a unique selector subsequence of interest downstream of the promoter sequence. The nucleic acids can each be transcribed to produce an RNA encoding the selector subsequence of interest by annealing a primer to the promoter sequence such that the promoter is recognized by an RNA polymerase and transcribing the nucleic acid with the RNA polymerase.

The solid support of each array in the library can optionally comprise any of the features of the solid support of the compositions described above. Optionally, the nucleic acids that make up the library can each comprise or encode any one or combination of features of the nucleic acids in the compositions described above.

The invention also provides a nucleic acid exon library that includes an array of nucleic acids, e.g., single-stranded nucleic acids, which can optionally be bound to a solid support. Each of the nucleic acids in the exon library comprises at least one upstream exon or exon subsequence and a processing feature subsequence. The processing feature subsequence facilitates interrogation of a target nucleic acid with the exon or exon subsequence to determine the sequence of a downstream exon sequence found in a target nucleic acid. The processing feature can optionally comprise a promoter that facilitates transcription of the nucleic acids of the array. Optionally, the processing feature can comprise or encode a restriction endonuclease recognition site.

Relatedly, the invention provides methods of determining a sequence of an exon-exon junction in a target nucleic acid. The methods include providing an array of nucleic acids each comprising an exon or an exon subsequence, producing one or more nucleic acid capture probes that each comprise or encode at least a portion of at least one of the exon subsequences from the array, and sequencing at least a portion of one or more target nucleic acids using one or more nucleic acid capture probes, thereby to determining the sequence of the exon-exon junction, e.g., that is present in an isolated target nucleic acid.

Sequencing one or more target nucleic acids can optionally comprise providing a population of nucleic acids and hybridizing the one or more nucleic acid capture probes to one or more target nucleic acids in the population that comprise a complementary target subsequence, thus producing at least one target nucleic acid-bound probe. The target nucleic acid-bound probe can be separated from unbound nucleic acids, and the recessed 3′ ends of strands of the target nucleic acid-bound probe can be extended with a DNA polymerase to produce a double-stranded fragment. Tags which comprise primer hybridization sites can be attached to the ends of the double stranded fragment, the tagged fragment can be transferred to a reaction volume that contains a mixture of sequencing reagents, and a sequencing reaction can be performed, e.g., to determine the sequence of the exon-exon junction.

Those of skill in the art will appreciate that the methods and compositions provided by the invention can be used alone or in combination. Systems that include modules for the production and/or sequencing of nucleic acid capture probes and/or target nucleic acids are also a feature of the invention. Such systems can optionally include detectors, array readers, excitation light sources, one or more output devices, such as a printer and/or a monitor to display results, and the like.

Kits are also a feature of the invention. The present invention provides kits that incorporate the compositions of the invention, optionally with additional useful reagents such as one or more enzymes that are used in the methods, e.g., an RNA polymerase, a DNA polymerase, a reverse transcriptase, etc., that can be packaged in a fashion to enable their use. Depending upon the desired application, the kits of the invention optionally include additional reagents, such as a control target nucleic acids, buffer solutions and/or salt solutions, including, e.g., divalent metal ions, i.e., Mg⁺⁺, Mn⁺⁺ and/or Fe⁺⁺, nucleic acid adapter tags, e.g., to prepare captured nucleic acids for sequencing, etc. Such kits also typically include a container to hold the kit components, instructions for use of the compositions, e.g., to practice the methods, and other reagents in accordance with the desired application methods, e.g., identifying transcription start sites, identifying exon-exon junctions, and the like.

### DEFINITIONS

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a nucleic acid capture probe” includes a combination of two or more nucleic acid capture probes; reference to “target nucleic acids” includes mixtures of target nucleic acids, e.g., each of which comprise a subsequence complementary to the target subsequence of a capture probe, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

An “array” is a physical or logical grouping of a set of elements/moieties (members) of interest (e.g., DNAs for transcription bound to solid supports, optionally in conjunction with transcription or other components relevant to the methods and compositions herein). A “physical array” is a set of specified array members arranged in a specified or specifiable spatial arrangement. A “logical array” is a set of specified array members arranged in a manner that permits specific and controlled access to the members of the set (as opposed to completely random access).

A “solid phase array” is an array in which the members of the array are fixed to a solid substrate. The fixation can be the result of any interaction that tends to immobilize components, including chemical linking, heat treatment, hybridization, ligand/receptor interactions, metal chelation interactions, ion exchange, hydrogen bonding, hydrophobic interactions and the like. A “solid substrate” has a fixed organizational support matrix, such as silica, polymeric materials, membranes, beads, pins, glass or other ceramics, etc. In some embodiments, at least one surface of the substrate is partially planar, but in others, the solid substrate is a discrete element such as a bead that can be dispensed into an organization matrix such as a microtiter tray.

A “liquid phase array” is an array in which the members of the array are free in solution, e.g., on a microtiter tray, or in a series of containers such as a set of test tubes or other containers. Most often, members of a liquid phase array are separated in space by subdividing the volume containing the members of the array into multiple discrete chambers such that each chamber contains less than a complete library of members, and ideally less than about 10% of the discrete members in the library. Such separation or fractionation of a population containing a plurality of unique sequences can be accomplished by sorting, dilution, serial dilution, and a variety of other methods.

A “constant region” refers to an invariable subsequence found in each of, e.g., a nucleic acid capture probe and/or a population transcribable nucleic acids that are tethered to a solid support. A constant region can optionally encode one or more features that can be useful in, e.g., preparing nucleic acid capture probes from RNAs that are transcribed from the tethered nucleic acids and/or preparing enriched target nucleic acids for resequencing, e.g., in a high-throughput sequencing system. Such features can include, e.g., one or more unique restriction endonuclease recognition sites, one or more unique primer hybridization sites, one or more affinity tags, and the like. The constant region permits the massively parallel preparation of nucleic acid capture probes from, e.g., RNAs produced by the transcription of the tethered nucleic acids and the massively parallel retrieval and isolation of, e.g., target nucleic acids from a nucleic acid sample of interest.

A “nucleic acid capture probe” is a single-stranded nucleic acid reagent that comprises a selector subsequence, a constant region, and a 5′ tag, e.g., affinity tag. The selector subsequence permits the nucleic acid capture probe to hybridize to and form a partially double-stranded structure with a target nucleic acid, e.g., a cDNA, an RNA, a fragment of a genomic DNA, and the like, that comprises a complementary target subsequence. The selector subsequence can also comprise one or more restriction endonuclease recognition sites that permit high-throughput preparation of a captured target nucleic acid for resequencing. The 5′ tag of a nucleic acid capture probe permits the isolation of the target nucleic acid of interest from a population of nucleic acids in a sample, e.g., via affinity purification. The constant region of a nucleic acid capture probe comprises features, e.g., unique restriction endonuclease recognition sites, primer hybridization sites, and the like, that permit high-throughput production of a nucleic acid capture probe from an RNA precursor and permit high-throughput preparation of an target subsequence, e.g., that has been isolated according to the methods of the invention, for resequencing, e.g., in a high-throughput system. For example, FIG. 4 illustrates capture probe 460, which comprises selector sequence 120, constant region 130, and 5′ tag 203.

A “processing module” is a system element (e.g., as in an automated system of the invention) that performs one or more steps in an overall system process, e.g., sequencing a target nucleic acid that has been isolated using the methods of the invention. For example, an automated “transcript processing module” optionally copies or transcribes nucleic acids tethered to a solid support, e.g., as described in the methods of the invention, while a “sequencing module” sequences target nucleic acids that have been prepared for sequencing, as described elsewhere herein.

A “selector sequence” is a subsequence present in a nucleic acid capture probe that permits the probe to hybridize specifically to those nucleic acids (“target nucleic acids”) in, e.g., a population of cDNAs, a population of mRNAs, a population of DNA fragments derived from a genomic DNA, and the like, that comprise a complementary subsequence (e.g., a “target subsequence”). In general, the selector subsequence of a capture probe can be custom designed to comprise any desired number of nucleotides, e.g., up to 100 nucleotides, more than 100 nucleotides, more than 250 nucleotides, or more than 500 nucleotides, in any desired sequence. As such, the selector subsequence of a capture probe determines the target nucleic acids that the probe can retrieve and the applications in which the probe can be beneficially used.

“Sufficiently double-stranded” is used herein in relation to a functional promoter region, e.g., a promoter region that is recognized by an RNA polymerase and from which an RNA polymerase can initiate transcription, that is formed when, e.g., a complementary oligonucleotide primer anneals to the one strand of a promoter sequence in a transcribable nucleic acid that is tethered to a solid support. Such a double-stranded region can be, e.g., at least 75% double-stranded, at least 80% double-stranded, at least 90% double-stranded, or more than 90% double stranded, and comprises promoter elements that are necessary and sufficient to permit an RNA polymerase to transcribe a template in vitro. The promoter region is sufficiently double-stranded when it is capable of being transcribed by an RNA polymerase.

A “tag” refers to a moiety linked to a nucleic acid of interest that can be used as a molecular recognition site to identify or distinguish the nucleic acid in a population, as a means to permit a protein, e.g., a DNA-binding protein, or an enzyme, e.g., an exonuclease, a restriction enzyme, a nicking enzyme, or the like, to recognize the nucleic acid and perform an activity, and/or as a means by which to separate the nucleic acid from the population. A tag can comprise one or more of a number of moieties, including labeled or modified nucleotides, e.g., biotinylated nucleotides. Tags can also comprise specific nucleotide sequences, e.g., restriction sites, cis regulatory elements, recognition sites for nucleic acid-binding proteins, sequences capable of forming secondary structures, or the like. The tags herein can optionally comprise one or more ligand, blocking group, phosphorylated nucleotide, phosphorothioated nucleotide, biotinylated nucleotide, digoxigenin-labeled nucleotide, methylated nucleotide, uracil, sequence capable of forming a hairpin structure, oligonucleotide hybridization site, restriction endonuclease recognition site, promoter sequence, sample or library identification sequence, and/or cis regulatory sequence.

A “target nucleic acid” is a nucleic acid that is desirably isolated from a population of nucleic acids using nucleic acid capture probe of the invention. A target nucleic acid comprises a “target subsequence”, which is complementary to the selector subsequence present in a capture probe, hybridizes to the capture probe, and permits the retrieval of a target nucleic acid from a population of nucleic acids, e.g., a population of RNAs, a population of cDNAs, a population of DNA fragments derived from a genomic DNA, and the like.

### BRIEF DESCRIPTION OF THE DRAWINGS

FIG. 1 schematically illustrates the synthesis of an RNA according to methods provided by the invention.

FIG. 2 schematically illustrates synthesis of a cDNA comprising a 5′ tag from the RNA depicted in FIG. 1.

FIGS. 3A and 3B schematically depict alternate methods of annealing a primer comprising a 5′ tag to an RNA.

FIG. 4 schematically depicts methods of producing a nucleic acid capture probe.

FIG. 5 schematically depicts alternate methods of producing a nucleic acid capture probe.

FIG. 6 schematically depicts methods of isolating a target nucleic acid from a population of nucleic acids in a sample with a capture probe.

FIGS. 7A and 7B schematically illustrate methods of preparing an isolated target nucleic acid for sequencing.

FIG. 8 schematically illustrates alternate methods of capturing a target nucleic acid.

FIG. 9 schematically illustrates the use of a capture probe to interrogate an mRNA or cDNA library to identify target nucleic acids that comprise novel exon-exon junctions.

FIG. 10 schematically illustrates the use of a capture probe to interrogate an mRNA or cDNA library to identify target nucleic acids that comprise alternate 3′UTR/polyA sites.

FIG. 11 schematically illustrates the use of a capture probe to interrogate an mRNA or cDNA library to identify target nucleic acids that were transcribed from alternate transcription start sites.

FIG. 12 schematically illustrates the use of a capture probe to interrogate the processing of miRNAs.

FIG. 13 depicts the results of an experiment performed to synthesize RNAs according to a method provided by the invention.

FIG. 14 schematically illustrates a method of preparing a target nucleic acid for sequencing.

FIG. 15 depicts the results of an experiment performed to show that GFP can be captured by the probes of the invention.

FIG. 16 shows the results of PCR reactions that were performed to determine the results of a pilot experiment using a capture probe comprising a target subsequence complementary to a subsequence of a luciferase mRNA.

FIG. 17 provides the results of experiments performed in Example 2.

FIG. 18 provides the results of a capture experiment described in Example 2.

### DETAILED DESCRIPTION

### Overview

Resequencing, or the targeted sequencing of one or more, e.g., candidate genes, transcripts, or genomic loci of interest, can advance the study of the relationship between, e.g., sequence variation and normal or disease phenotypes. By targeting nucleic acid sequencing efforts to, e.g., specific regions of large (e.g., human) genomes or specific genes, resequencing can be a labor saving, cost-efficient technique by which to perform genotype analyses or genetic variation analyses. However, one of the major challenges in resequencing efforts is the efficient isolation of specific nucleic acid targets of interest. The methods and compositions provided by the invention can be advantageously used to enrich nucleic acids comprising subsequences of interest from a population of nucleic acids e.g., a population of cDNAs, a population of mRNAs, a population of DNA fragments derived from a genomic DNA, and the like, in preparation for targeted resequencing,

Currently, sequences of interest in, e.g., a population of cDNAs, fragments of a genomic DNA, or the like, can be selectively enriched for resequencing, e.g., in a high-throughput sequencing system, through a labor-intensive process whereby nucleic acid fragments comprising the desired sequence(s) are each amplified via PCR. However, the amplification of, e.g., multiple genes or genomic loci, entails the parallel design, optimization, and execution of up to, e.g., thousands of individual PCR reactions, representing a substantial investment in time, effort, and money. In addition, preparing samples for resequencing via PCR can be technically challenging if enriching a locus that comprises the sequence of interest requires the amplification of nucleic acid fragments that are longer than a few hundred kilobases. Furthermore, PCR can only be used to amplify fragments that are known to comprise the sequence(s) of interest, precluding the discovery of, e.g., additional genes, genomic loci, or the like, that also comprise such sequence(s).

In contrast, the methods and compositions provided by the invention (“GREPSEQ”), can be used to produce custom nucleic capture probes that “grep” (e.g., search) a population of nucleic acids for those nucleic acid species that comprise a particular subsequence (“seq”), e.g., a “target subsequence”. Producing the capture probes according to the methods of the invention eliminates the cost, labor, and infrastructure that is typically required for large-scale PCR experiments. The compositions used in the synthesis of the capture probes are reusable, which can further reduce the cost of isolating desirable nucleic acids for resequencing. The invention also provides methods of using said nucleic acid capture probes in the efficient capture and recovery of nucleic acids, e.g., “target nucleic acids”, that comprise a sequence of interest from a population of nucleic acids. Unlike PCR, the capture probe strategy described herein does not bias against, e.g., uncharacterized genes, transcripts, genomic loci, or the like, that, comprise one or more sequences of interest. The specificity of the capture can be optimized by varying the selector subsequence of the capture probe and/or by varying the stringency of the hybridization conditions, as described elsewhere herein.

For ease of discussion, the present invention will be described with schematic illustrations of the compositions and methods it provides. Next, details regarding sequencing reactions, high-throughput sequencing systems, and downstream applications in which the compositions and methods of the invention can be beneficially used are described. Further details regarding, enzymes, nucleic acids, kits, and broadly applicable molecular biological techniques that can be used to perform the methods are described thereafter.

Methods and Compositions for the Synthesis of Nucleic Acid Capture Probes

One of the major challenges of targeted resequencing is the efficient isolation of nucleic acids of interest to sequence, e.g., in a high-throughput sequencing system. In one aspect, the invention provides compositions and methods useful for the efficient, scalable, low-cost production of nucleic acid capture probes that can be beneficially used in the isolation of nucleic acids that comprise one or more subsequences of interest (“target subsequences”) from, e.g., a population of nucleic acids, such as a population of cDNAs, a population of mRNAs, a population of DNA fragments derived from a genomic DNA, and the like. The methods of producing capture probes provided herein entail the synthesis of RNAs from tethered nucleic acids and the subsequent synthesis of cDNAs from the RNAs.

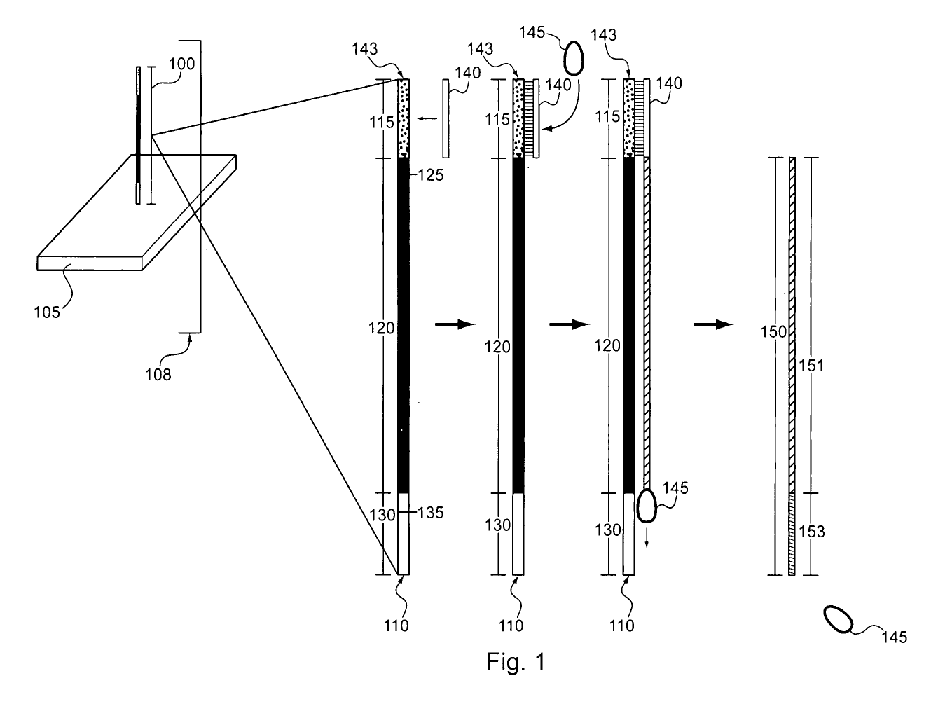

RNAs are synthesized as schematically illustrated in FIG. 1. Nucleic acid 100 is tethered to solid support 105 by 5′ end 110. The solid support can optionally comprise a bead, a slide, or the like, as described elsewhere herein. Nucleic acid 100 comprises a single strand of a promoter sequence 115, e.g., a promoter sequence that is recognized by an RNA polymerase. Nucleic acid 100 also includes selector subsequence of interest 120 downstream of promoter 115 and includes a first strand of unique restriction endonuclease recognition site 125. Nucleic acid 100 also includes constant region 130, which comprises processing features, e.g., one strand of unique restriction endonuclease recognition site 135, which facilitates the production of a nucleic acid capture probe. Constant region 130 can optionally comprise one or more other features that facilitate sequencing of, e.g., a nucleic acid capture probe or a sequence complementary to the capture probe. (These features and their uses are described in further detail below.)

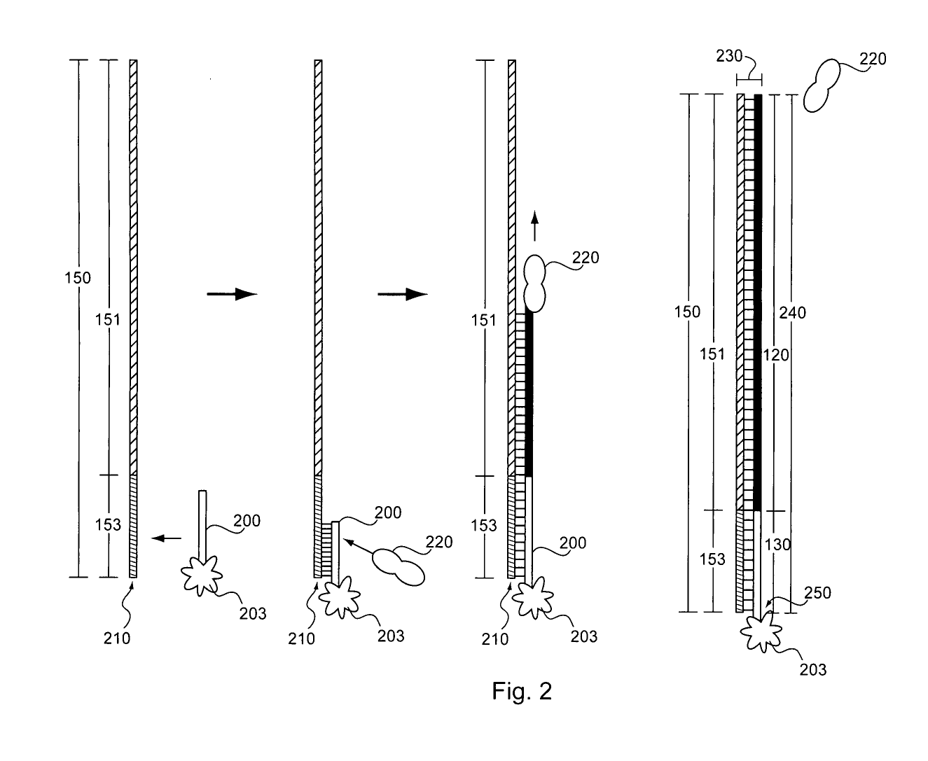

Nucleic acid 100 can be transcribed when primer 140 is annealed to the promoter sequence at 3′ end 143 to produce an RNA polymerase recognition site that is double-stranded, e.g., sufficiently double stranded, to permit recognition and subsequent transcription of nucleic acid 100 by RNA polymerase 145. Polymerase 145 can travel toward 5′ end 110 of nucleic acid 100, transcribing selector subsequence 120 and constant region 130 to produce RNA 150, which comprises transcribed selector subsequence 151 and transcribed constant region 153. The repeated transcription of nucleic acid 100 amplifies the number of RNA molecules, e.g., RNA 150 molecules, thus increasing the yield of capture probes that are then produced from the RNAs in the following steps of the methods. RNA 150 can be subsequently used to synthesize a cDNA, e.g., cDNA 240 (see FIG. 2), in preparation to produce a nucleic acid capture probe. The reverse transcription of cDNAs, e.g., cDNAs 240 in FIG. 2, from the RNAs, e.g., RNAs 150, amplifies the yield of cDNAs, thus further amplifying the yield of capture probes that are then produced from the cDNAs, as described below.

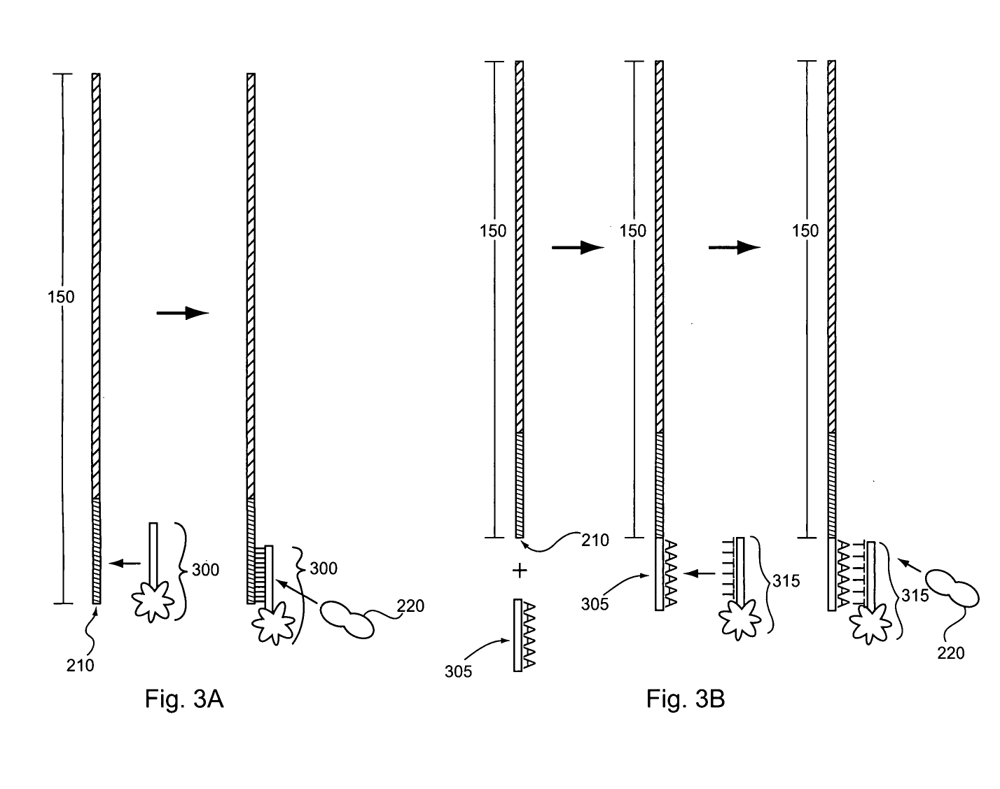

To synthesize cDNAs from RNA 150, primer 200 (see FIG. 2), which comprises 5′ tag 203, can be annealed to 3′ end 210 of RNA 150 produced, e.g., by the method depicted in FIG. 1 and described above. Primer 200 can be extended by reverse transcriptase 220 to generate RNA:DNA duplex 230, which comprises RNA 150 and cDNA strand 240 with tag 203 at 5′ end 250. cDNA 240 includes selector subsequence 120 and constant region 130, which are synthesized during reverse transcription of transcribed selector subsequence 151 and transcribed constant region 153 of RNA 150. In certain embodiments, primer 200 can include a constant region if nucleic acid 100, from which RNA 150 is transcribed, does not already include a constant region.[0067] Any of a number of methods can be used to anneal a tagged primer to the 3′ end of RNA 150 in preparation for the synthesis of cDNAs, e.g., cDNA 240. In one embodiment, depicted in FIG. 3A, tagged primer 300 can comprise a sequence complementary to that of 3′ end 210. Alternately, as shown in FIG. 3B, polyA tail 305 can be enzymatically added, e.g., by a polyA polymerase, a terminal transferase, or an RNA ligase, to 3′ end 210 of RNA 150. In an alternate embodiment, a different constant region can be attached, e.g., to RNA 150, using, e.g., an RNA ligase. Following the addition of polyA tail 305, or other constant sequence, to RNA 150, tagged polyT primer 315, or a primer complementary to the newly-added constant sequence, can be annealed to polyA tail 305 and extended with reverse transcriptase 220 to produce an RNA: DNA duplex that comprises a cDNA strand with a tagged 5′ end, as described above.

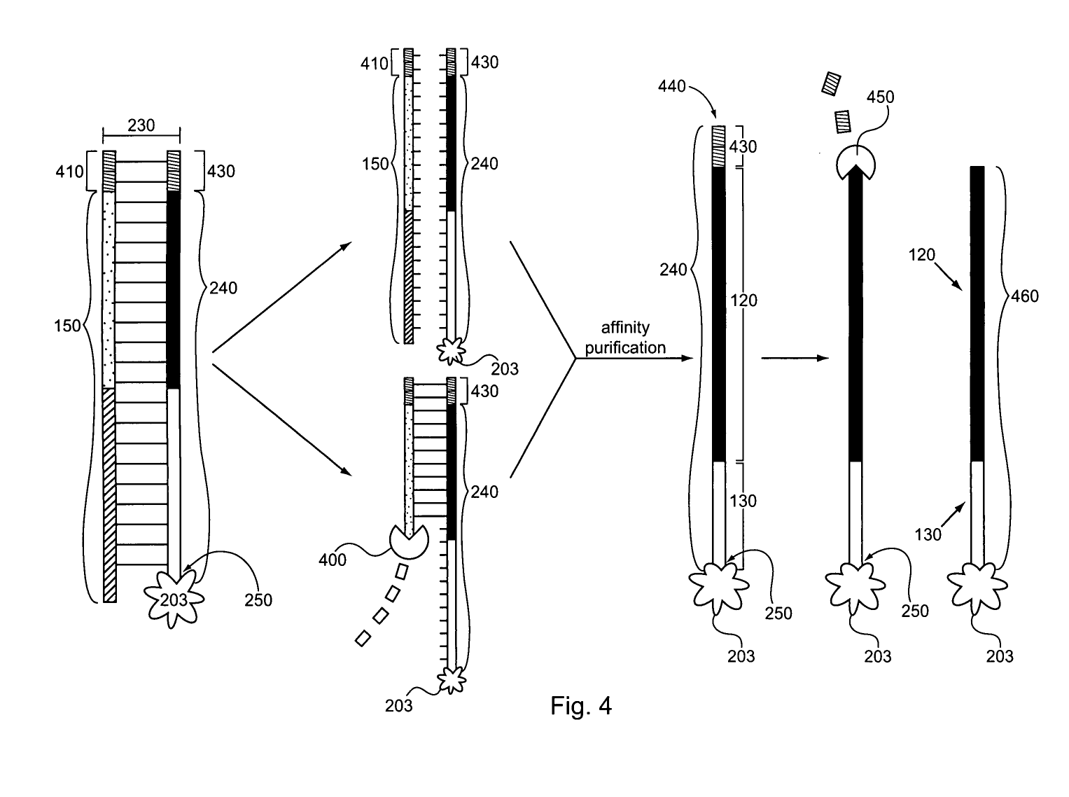

One embodiment of the methods of producing a nucleic acid capture probe from RNA:DNA duplex 230 is schematically illustrated in FIG. 4. In this embodiment, RNA strand 150 and cDNA strand 240 are separated, e.g., via denaturation or via digestion of RNA strand 150 with RNAse H400. Following denaturation or digestion, cDNA 240 can be recovered, e.g., via affinity purification using tag 203 at 5′ end 250.

Empirically, it has been observed that transcription of RNA 150 (see, e.g., FIG. 1 and the corresponding description) from nucleic acid 100 by RNA polymerase 145 can result in the addition of one or more nucleotides 410, e.g., nucleotides that are not encoded in transcribed nucleic acid 100, to the 5′ end of RNA 150 (see FIG. 4). Accordingly, during the reverse transcription of RNA 150 to produce tagged cDNA 240, one or more nucleotides 430 are added to the 3′ end of cDNA 240, e.g., the end of the cDNA that typically encodes selector subsequence 120. To ensure the most efficient hybridization of a target subsequence, e.g., present in a target nucleic acid, to selector subsequence 120, e.g., in downstream applications, it is beneficial to remove nucleotides 430 from the 3′end during the synthesis of nucleic acid capture probes. In one embodiment, nucleotides 430 are removed from 3′ end 440 of cDNA 240 by limited digestion with enzyme 450, which comprises a 3′→5′ exonuclease activity, to produce nucleic acid capture probe 460. Capture probe 460 comprises selector subsequence 120, which can hybridize to complementary target subsequences present in target nucleic acids, constant region 130, and 5′ tag 203, which, in certain embodiments, can comprise an affinity tag. In an alternate and preferred embodiment, an endonuclease that cuts upstream of nucleotides 430, e.g., a type III restriction endonuclease, can be used to remove these extra nucleotides (e.g., before strands 150 and 240 are separated).

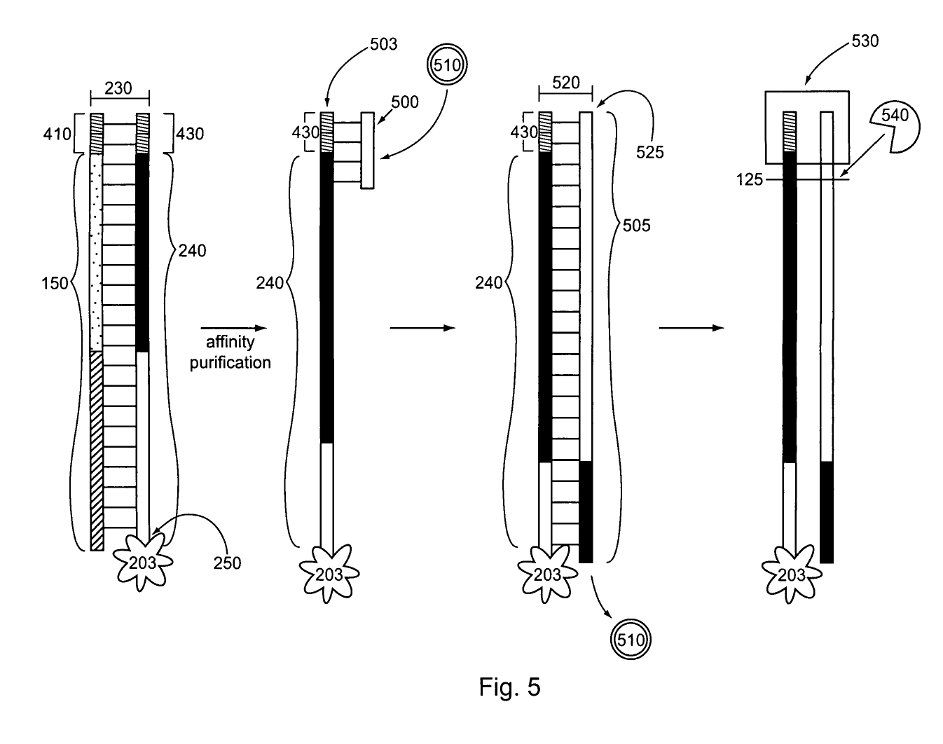

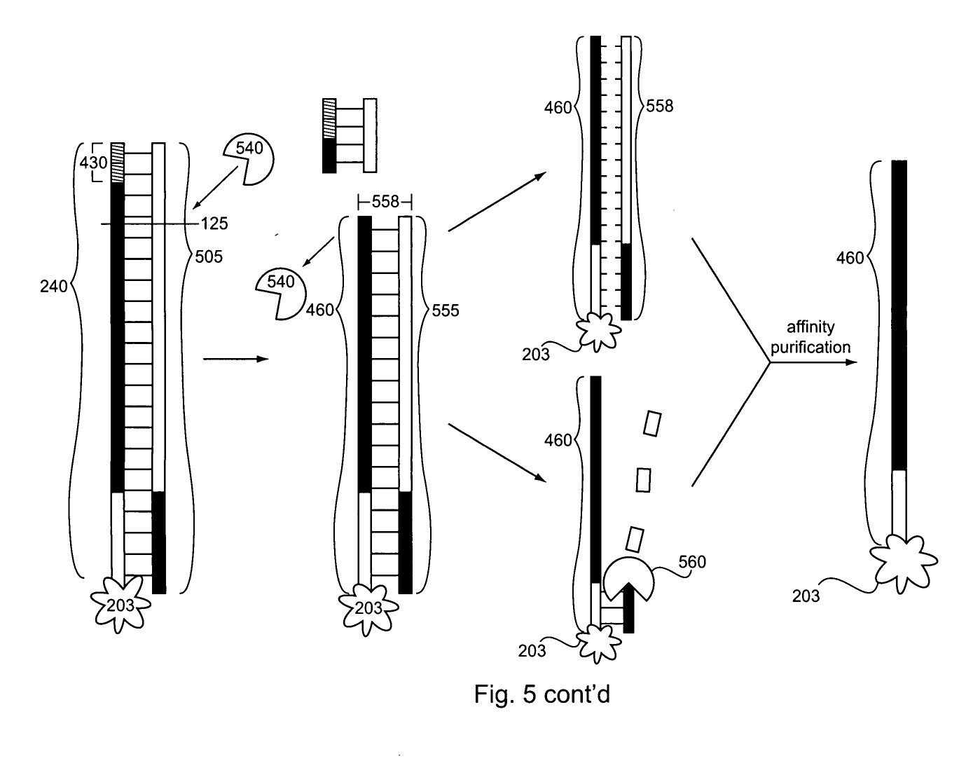

A nucleic acid capture probe can be produced using an alternate method that includes the synthesis of a double-stranded cDNA intermediate (see FIG. 5). This method also begins with the synthesis of an RNA and the subsequent reverse transcription of the RNA, as described above, to produce RNA:DNA duplex 230, which comprises RNA 150 and cDNA 240 with tag 203 at 5′ end 250. In this alternate method, untagged primer 500 is annealed to 3′ end 503 of purified cDNA 240 and extended with DNA polymerase 510 to produce a double-stranded cDNA 520, which comprises cDNA 240 and complementary strand 505. Double-stranded cDNA intermediate 520 is produced in preparation to remove the extra nucleotides that are added to the 5′ end of RNA 150 during transcription of tethered nucleic acid 100 and to the 3′ end of cDNA 240 during the reverse transcription of RNA 150, as described previously.

Although strand 505 can be synthesized by reverse transcriptase 220, e.g., using strand 240 as a template, it has been empirically observed that second strand cDNA synthesis by reverse transcriptase 220 can result in truncations at strand 505's 5′ end 525. Such truncations can prohibit the removal of the extra nucleotides at untagged end 530 of double-stranded cDNA 520 by digestion with restriction endonuclease 540. For the reasons elaborated above, e.g., to promote efficient hybridization of a target nucleic acid to selector subsequence 120 in downstream applications, it is beneficial to remove the one or more extra nucleotide base pairs from untagged end 530 of double-stranded cDNA 520. Thus, extending primer 500 with DNA polymerase 510 to produce strand 505 is preferred.

The extra nucleotides at untagged end 530 of double-stranded cDNA 520 are removed by digesting 520 with restriction endonuclease 540, which recognizes site 125 (see, e.g., FIG. 1), a restriction site proximal to the untagged end of double-stranded cDNA 520. Following digestion by restriction endonuclease 540, nucleic acid capture probe 460 and untagged strand 555 of double-stranded digested cDNA 558 are separated, e.g., via denaturation or via digestion with enzyme 560. Enzyme 560 has a 5′→3′ exonuclease activity and will specifically digest strand 555 of double-stranded digested cDNA 558. Following digestion (or denaturation), nucleic acid capture probe 460 can be recovered, e.g., via affinity purification using tag 203.

One benefit of synthesizing nucleic acid capture probes using RNA and cDNA intermediates is that composition 108 (see FIG. 1), e.g., nucleic acid 100 tethered to solid support 105, e.g., from which RNA 150 is transcribed, can be reused, e.g., in up to 1000 rounds of solid-phase transcription, more preferably up to 10,000 rounds of solid-phase transcription, or, most preferably, up to 50,000 rounds of solid-phase transcription. Compositions provided by the invention, e.g., composition 108, can thus renewably supply reagents from which nucleic capture probes are then produced. This can advantageously reduce the cost, labor, and infrastructure that are typically required for sample preparation using large-scale PCR.

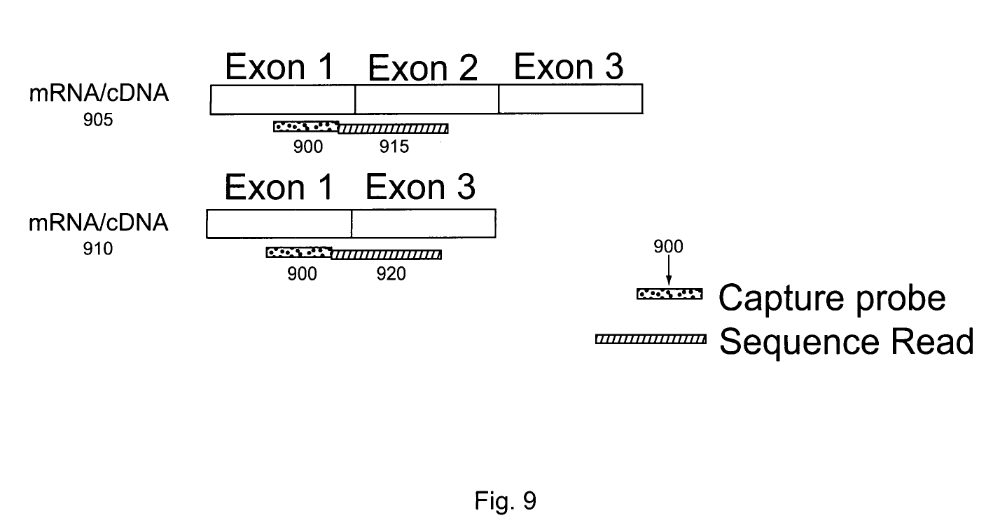

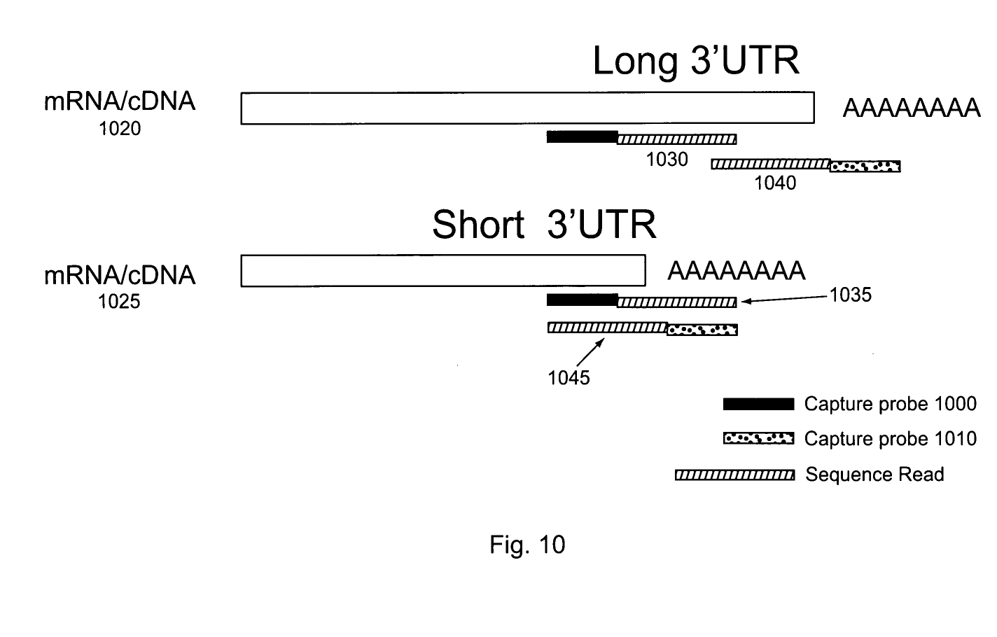

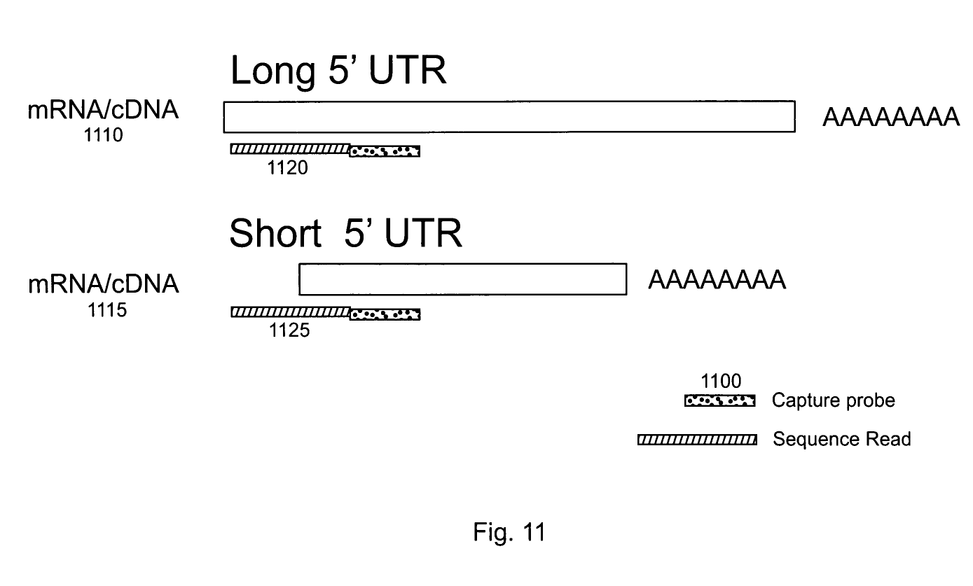

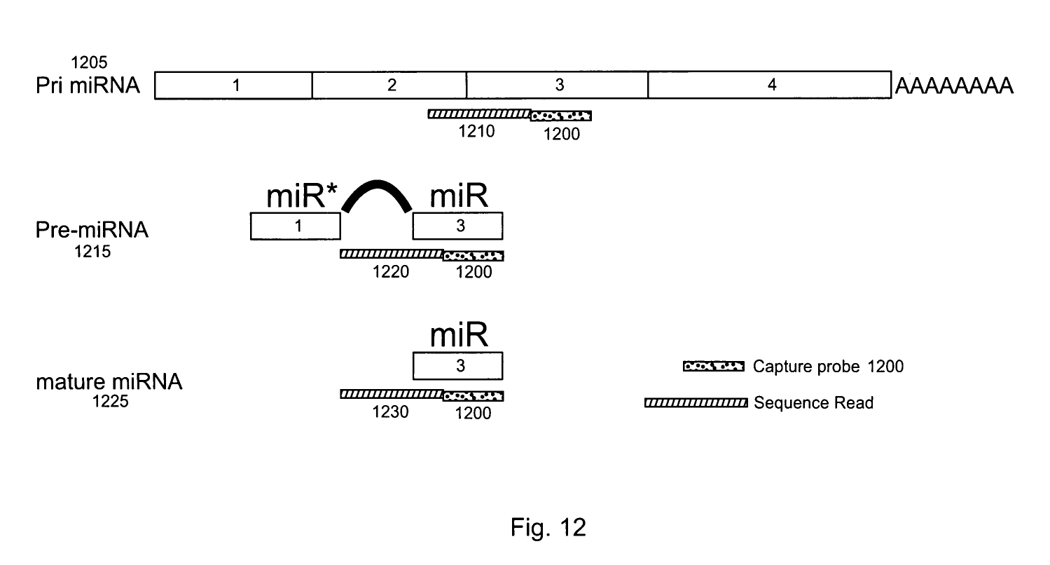

Nucleic acid capture probes, e.g., synthesized according to any one or combination of the methods described above, can optionally comprise any of a variety of selector subsequences. As such, capture probes are adaptable for use in a variety of downstream applications, including, but not limited to, e.g., the interrogation of alternative splicing libraries to capture target nucleic acids with a novel exon-exon junction (depicted schematically in FIG. 9), the interrogation of mRNA libraries to capture target nucleic acids that comprise alternative 3′UTR/polyA sites (depicted schematically in FIG. 10) or alternative transcription start sites (depicted schematically in FIG. 11), the interrogation an miRNA processing library capture target nucleic acids that are produced by miRNA processing steps (depicted schematically in FIG. 12), and others. Specific details regarding sample interrogation techniques, and DNA resequencing techniques used to analyze “captured” target nucleic acids are described elsewhere herein, as are the details of downstream applications, e.g., as schematically illustrated in FIGS. 9-12, in which capture probes can be beneficially used.

Accordingly, compositions provided by the invention, e.g., compositions from which capture probes can be produced, can be advantageously designed to comprise a plurality of nucleic acids. For example, a composition provided by the invention can comprise a solid support to which, e.g., up to 400,000 nucleic acids, up to 1,000,000 nucleic acids, or, in some embodiments, up to 10,000,000 nucleic acids can be tethered, e.g., via their 5′ ends. The plurality of nucleic acids tethered to a solid support can represent any number of unique selector subsequences, e.g., 1 unique selector subsequence per 1000 tethered nucleic acids, 1 unique selector subsequence per 100 tethered nucleic acids, or even 1 unique selector subsequence per tethered nucleic acid.

This feature of the compositions beneficially permits the simultaneous production of numerous capture probe variants that can be used for the highly efficient, highly parallel “capture” of, e.g., up to 1 million target nucleic acids, up to 100 million target nucleic acids, or up to 1 billion target nucleic acids, from a nucleic acid sample. A population of capture probes in which at least 10,000 unique selector subsequences, at least 100,000 unique selector subsequences, or at least 1 million unique selector subsequences are represented can be simultaneously produced from a library provided by the invention. Details regarding libraries comprising one or more arrays, e.g., from which a plurality of capture probe variants can be produced, are discussed in further detail elsewhere herein.

As described above, another advantageous aspect of the methods and compositions described herein is that iterative cycles of transcription, e.g., of the nucleic acids tethered to a solid support, effectively amplifies the population of RNAs from which capture probes can be produced. Such amplification can increase the number of capture probes produced by the methods and, thereby, improve the efficiency with which rare target nucleic acids, e.g., low copy RNAs, low copy cDNAs, or rare alleles in a genome, are retrieved from a nucleic acid sample by the capture probes. Promoter sequences and RNA polymerases that are most beneficially used with the methods of, e.g., synthesizing an RNA and/or producing a capture probe, are elaborated elsewhere herein.

Methods and Compositions for the Targeted Enrichment and Sequencing of Nucleic Acids of Interest from a Population of Nucleic Acids

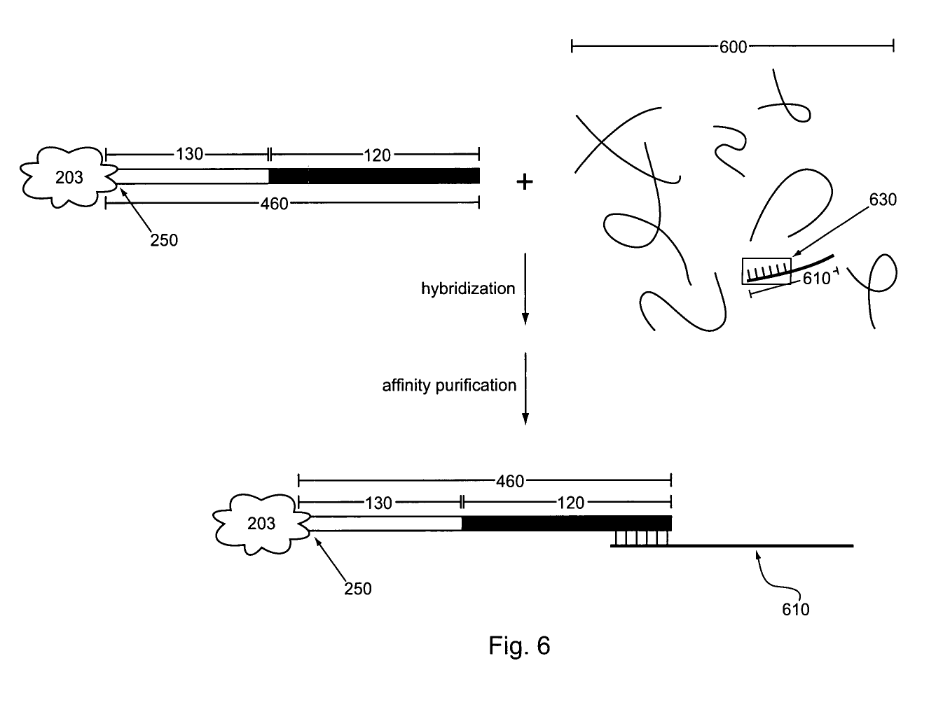

In a related aspect, the invention provides methods and compositions that facilitate the isolation of nucleic acids of interest from a population of nucleic acids. The capture probe, e.g., synthesized according to the methods described above, can be introduced into a nucleic acid sample, e.g., a population of cDNAs, a population of mRNAs, a population of miRNAs, a population of DNA fragments derived from a genomic DNA, or the like. The probes then hybridize with a subset of nucleic acids in the sample, e.g., those nucleic acids that comprise a “target subsequence”, e.g., a subsequence complementary to the selector subsequence of the capture probe (see FIG. 6). Following the purification of the desired nucleic acids (e.g., “target nucleic acids”) from the sample, these selectively enriched nucleic acids can then be prepared for resequencing, e.g., using a high-throughput sequencing system, as described elsewhere herein. Thus, the selector subsequence of a capture probe, e.g., selector subsequence 120 of capture probe 460, most advantageously comprises a sequence complementary to that present in the target nucleic acid(s) of interest that are to be enriched from the sample.

Methods of isolating a target nucleic acid from a nucleic acid sample are schematically detailed in FIG. 6. First, capture probe 460 is introduced to nucleic acid sample 600, which comprises target nucleic acid 610. Selector subsequence 120 of capture probe 460 hybridizes to a complementary target subsequence 630 of target nucleic acid 610. In preferred embodiments, the hybridization is performed in a liquid phase. Capture probe 460, and target nucleic acid 610 (to which probe 460 is hybridized), can then be separated from the other nucleic acids in sample 600 via affinity purification using tag 203 at 5′ end 250 of capture probe 460. In certain embodiments, capture probes in which at least 10,000 unique selector subsequences, at least 100,000 unique selector subsequences, or at least 1 million unique selector subsequences are represented can be used to simultaneously “grep” (or search) a nucleic acid sample for target nucleic acids of interest.

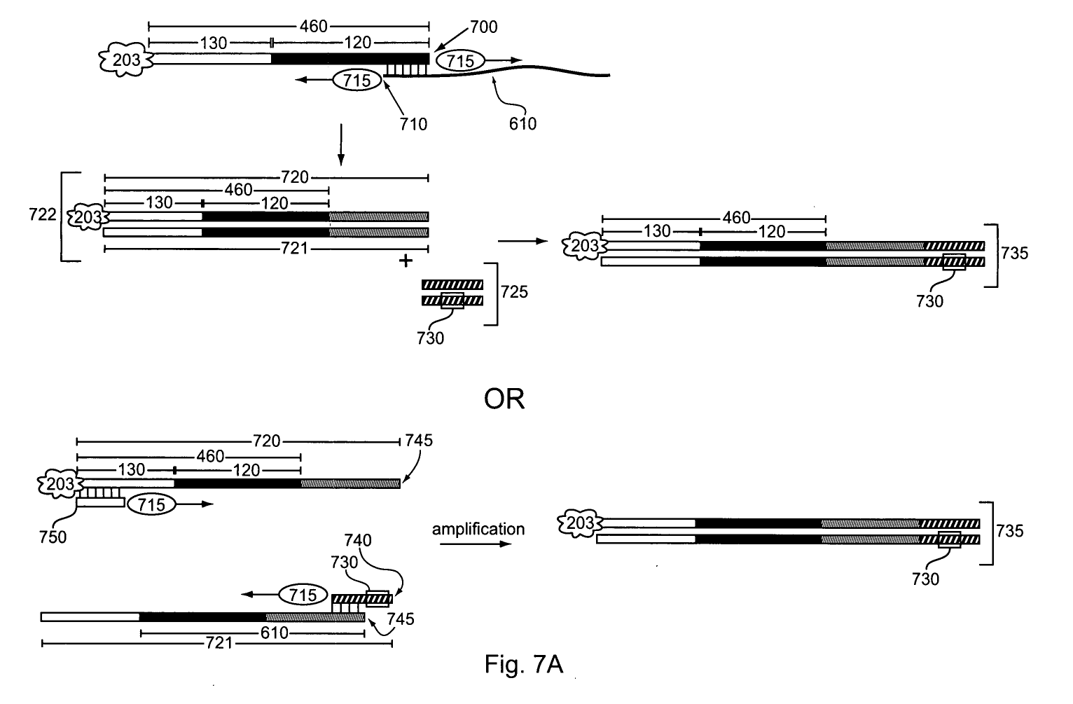

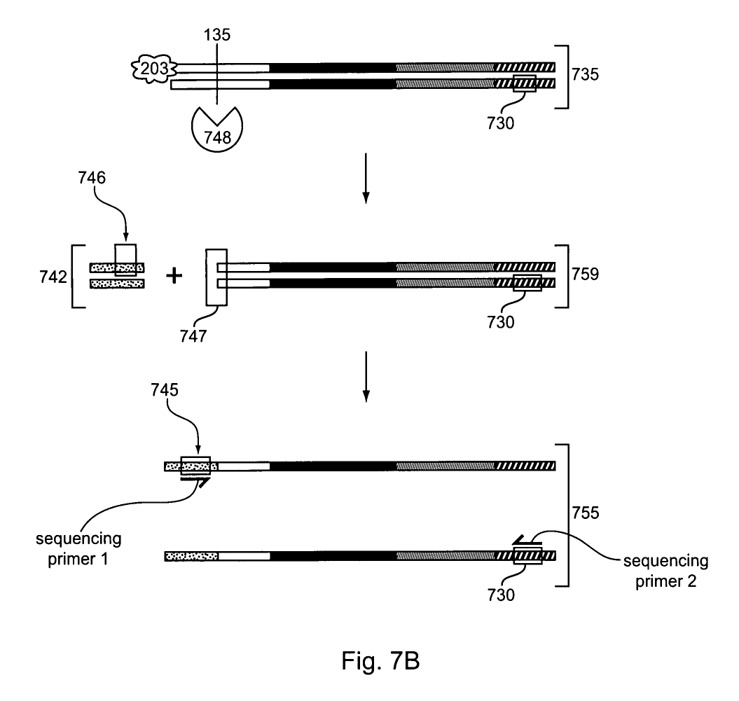

To prepare target nucleic acid 610 for resequencing, recessed 3′ end 700 (see, e.g., FIG. 7) of capture probe 460 and recessed 3′ end 710 of target nucleic acid 610 can be extended, e.g., with a DNA polymerase 715 to produce double-stranded fragment 722. Double-stranded nucleic acid adapter 725, which comprises sequencing primer hybridization site 730, can be attached to the untagged end of fragment 722 to produce fragment 735. Alternately, fragment 735 can be produced by hybridizing oligonucleotide 740, which comprises sequencing primer hybridization site 730, to 5′ end 745 of strand 721, which comprises sequences corresponding to target nucleic acid 610. Oligonucleotide 750 can be hybridized to a subsequence that corresponds to constant region 130 of capture probe 460 in strand 720. Hybridized oligonucleotides 740 and 750 can be extended with polymerase 715 to produce fragment 735.

Following removal of tag 203 from fragment 735 by digestion of unique restriction endonuclease site 135 (see, e.g., FIG. 1 and the corresponding description) by restriction endonuclease 748, double-stranded nucleic acid adapter 742, which comprises sequencing primer hybridization site 746, can be attached to end 747 of tagless fragment 759 using strategies described above. Resulting fragment 755 can then be sequenced by any one of a variety of high-throughput sequencing systems, e.g., single molecule sequencing systems, bioluminometric sequencing systems, and others, as will be discussed elsewhere herein.

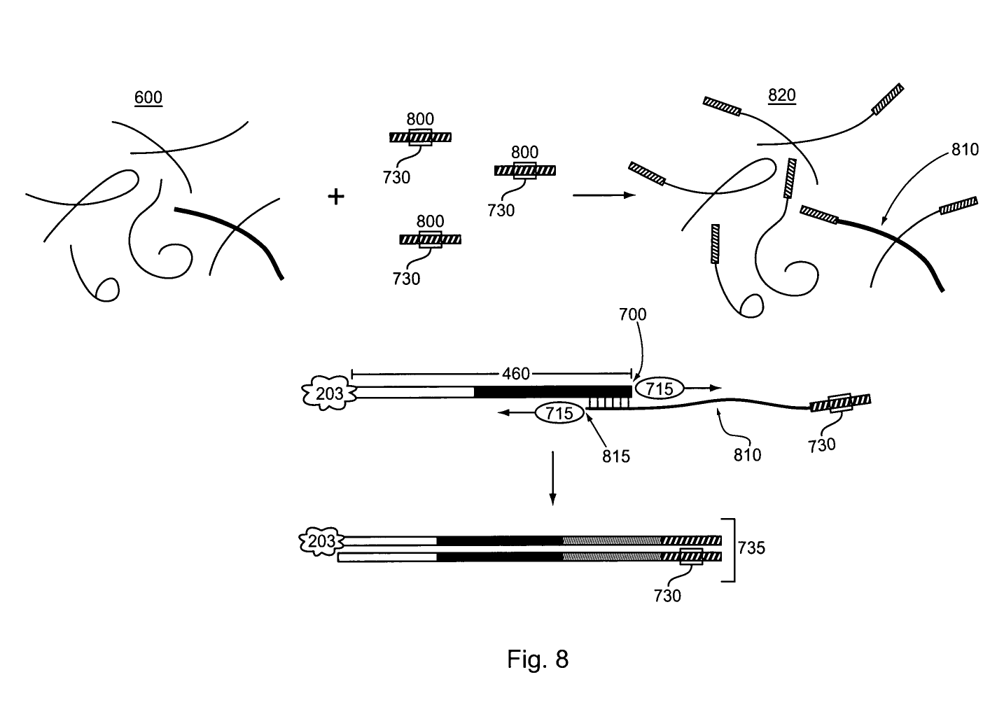

In an alternate embodiment (see FIG. 8), oligonucleotide 800, comprising primer hybridization site 730, can be attached to the 5′ ends of the nucleic acids in sample 600, e.g., via the hybridization or ligation of random hexamer sequences in oligonucleotide 800 to the nucleic acids in sample 600, to produce tagged sample 820. Following capture of target nucleic acid 810 by capture probe 460, as described previously, recessed 3′ end 700 of capture probe 460 and recessed 3′ end 815 of target nucleic acid 810 can be extended with DNA polymerase 715 to produce double-stranded fragment 735. Tag 203 is removed via endonuclease digestion, as described above (see FIG. 7B), and double-stranded nucleic acid adapter 742, which comprises primer hybridization site 746, can be attached to end 747 of tagless fragment 759. Resulting fragment 755 can then be sequenced, as described above.

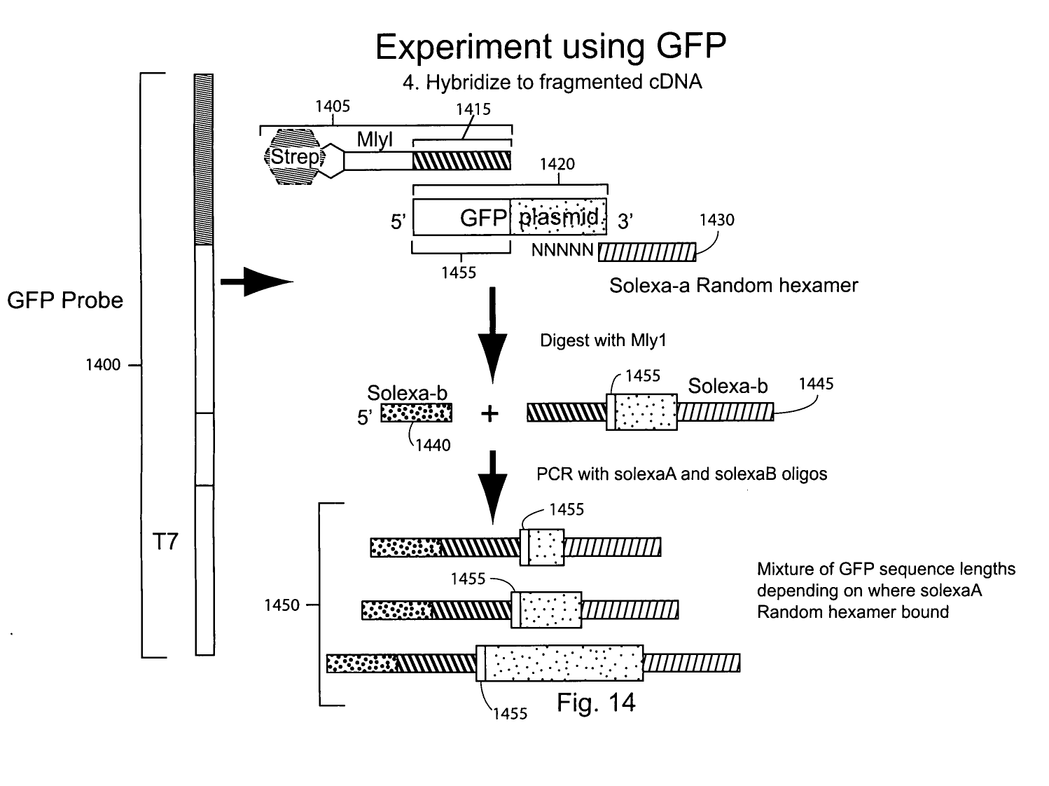

A general schematic summary of the steps for preparing a target nucleic acid for sequencing is provided in FIG. 14. Nucleic acid 1400, which, like nucleic acid 100 (See FIG. 1 and corresponding description) comprises a constant region, a selector subsequence, and a single strand of a promoter sequence, can be transcribed and processed to produce capture probe 1405. Capture probe 1405 comprises selector subsequence 1415, which is complementary to target subsequence 1455 present in target nucleic acid 1420. Target nucleic acid 1420 can then be processed, e.g., prepared for sequencing, by, e.g. extending random hexamer 1430 to produce fragment 1445. Tag 1440 can be attached to fragment 1445, using any of the methods described previously, and the resulting tagged fragements 1450 can be sequenced, e.g., in a high-throughput sequencing system, to determine the nucleotide sequence of nucleic acid subsequence 1455, which is a subsequence of the target nucleic acid to which selector subsequence 1415 hybridized.

### Further Details Regarding the Features of Capture Probes

Selector Subsequence

Nucleic acid capture probes, e.g., synthesized according to any one or combination of methods provided herein (See, e.g., FIG. 1 through FIG. 5 and the corresponding description), can be used to purify specific target nucleic acids comprising sequences of interest (“target subsequences”) from a sample population in an efficient, cost effective manner that does not entail labor-intensive, time-consuming cloning techniques (see, e.g., FIGS. 6, 8 and corresponding description). Typically, a nucleic acid capture probe of the invention (see, e.g., capture probe 460 in FIG. 4) includes a selector subsequence (see, e.g., selector subsequence 120 in FIG. 4), e.g., a capture motif that can hybridize to a complementary target subsequence in a target nucleic acid in a nucleic acid sample, e.g., a population of mRNAs, cDNAs, fragments derived from a genomic DNA, or the like. The selector subsequence that is included in a capture probe delimits the downstream applications in which a particular capture probe can be most beneficially used, as described elsewhere herein. Nevertheless, the nucleotide sequence of the selector subsequence of a capture probe is not particularly limiting.

Because methods of isolating one or more target nucleic acids with a capture probe rely on the efficient hybridization of a capture probe's selector subsequence to a complementary target subsequence in a target nucleic acid, a capture probe of the invention is most advantageously single-stranded. Due to the thermodynamic stability of homoduplex DNA, double-stranded DNA that has been denatured can rapidly re-anneal, and such re-annealing can reduce the efficiency with which, e.g., a capture probe can hybridize to a target subsequence, and impede the isolation and purification of a target nucleic acid from a sample of interest. Using single-stranded nucleic acid capture probes to enrich specific sequences, e.g., for downstream analysis, from a nucleic acid sample eliminates these issues.

The selector subsequence of a capture probe can include one or more processing features, e.g., one or more unique restriction endonuclease recognition site, which can be useful, e.g., in the production of a capture probe from an RNA (see, e.g., restriction endonuclease recognition site 125 in FIG. 5A, FIG. 5B, and the corresponding description). A restriction endonuclease recognition site can be beneficially used in the removal extra nucleotides (e.g., nucleotides 430 in FIG. 5B) present at the 3′ end of a capture probe, which nucleotides can impede the hybridization of the selector subsequence in a capture probe to a complementary target subsequence of interest in a target nucleic acid present in, e.g., a population of nucleic acids.

Constant Region

In general, a capture probe can also include a constant region (see, e.g., constant region 130 in FIG. 4), which, like the selector subsequence, can encode one or more features that are useful in, e.g., the production of a capture probe from an RNA or in the preparation of a captured target nucleic acid for resequencing, e.g., using a high-throughput sequencing system (see, e.g., FIG. 7). Such features can include one or more restriction endonuclease recognition site (see, e.g., restriction endonuclease recognition site 135 in FIG. 7B and corresponding description), which can be useful in removing the affinity tag from a capture probe to which a target nucleic acid has hybridized in preparation for sequencing (see, e.g., FIGS. 7A, 7B and the corresponding description). Additionally or alternatively, the constant region can optionally comprise a oligonucleotide hybridization site, e.g., to permit the annealing of a primer in preparation for, e.g., a sequencing reaction, an amplification reaction, a second-strand cDNA synthesis reaction, and/or the like.

Tags

In preferred embodiments, 5′ end of a nucleic acid capture probe is typically attached to a tag, e.g., a labeled and/or modified nucleotide. Such tags can also comprise specific nucleotide sequences, e.g., restriction sites, cis regulatory elements, recognition sites for nucleic acid-binding proteins, sequences capable of forming secondary structures, sequences that can be recognized by an antibody, and/or the like. The tags herein can optionally comprise one or more ligand, affinity ligand, blocking group, phosphorylated nucleotide, phosphorothioated nucleotide, biotinylated nucleotide, digoxigenin-labeled nucleotide, methylated nucleotide, uracil, inosine, sequence capable of forming a hairpin structure, oligonucleotide hybridization site, restriction endonuclease recognition site, promoter sequence, sample or library identification sequence, and/or cis regulatory sequence.

Tags that are ideally attached to the 5′ end of a capture probe can be beneficially used in affinity purification methods. Thus, the tag attached to the 5′ of a capture probe can be advantageously used to selectively isolate a target nucleic acid that is hybridized to the capture probe from other nucleic acids in a sample population. Similarly, the tag can be used to retrieve capture probe precursors from reverse transcriptase reactions, amplification reactions, restriction digestion reactions, etc., that are performed to produce a capture probe, e.g., from an RNA transcribed from a nucleic acid that has been tethered to a solid support. Such tags include biotinylated nucleotides, nucleotide sequences that can be specifically recognized by, e.g., an antibody, another protein, and/or the like. (Methods of attaching a tag to a capture probe are described elsewhere herein).

In preferred embodiments, tags attached to target nucleic acids, e.g., isolated according to the methods herein, comprise one or more oligonucleotide hybridization sites, e.g., to permit the hybridization of sequencing primers so that the target nucleic acids can be sequenced, e.g., using a high-throughput sequencing system. Additionally or alternatively, the tags attached to a target nucleic acid can comprise one or more unique DNA sequence identifiers, which can be useful in multiplexed sequencing. For example, target nucleic acids, e.g., to which tags comprising DNA sequence identifiers have been attached, from independent samples can be optionally pooled and sequenced, and the presence of DNA the sequence identifiers in tags can facilitate the segregation and characterization of the target nucleic acids, e.g., following sequencing.

### High-Throughout Nucleic Acid Sequencing Systems

DNA sequencing refers to methods for determining the order of the nucleotide bases, e.g., adenine, guanine, cytosine, and thymine, in a molecule of DNA, such as a target nucleic acid. Typically, a sequencing reaction mix includes a polymerase; adenine, guanine, cytosine, and thymine nucleotides; a template strand, an oligonucleotide primer that comprises a sequence complementary to a sequence in the template strand, and a divalent cation, e.g., Mn²⁺ or Mg²⁺, which improves the polymerase's activity. In general, a sequencing reaction entails annealing the oligonucleotide primer to the single-stranded DNA template and extending the primer with the polymerase, which incorporates nucleotide bases into a nascent chain to synthesize a DNA molecule whose sequence is complementary to that of the template strand. If a double-stranded template is provided, it is denatured prior to the annealing and extension steps. During synthesis, the incorporation of each individual nucleotide is detected, permitting the determination of the pattern of adenines, guanines, cytosines, and thymines in the template strand. In the present invention, determining the order of nucleotides in, e.g., a target nucleic acid that has been isolated according to the methods described herein, can be useful in, e.g., genotyping, variation analysis, identifying exon-exon junctions, identifying alternative transcription start sites, identifying alternative polyadenylation sites, and other downstream applications.

One sequencing method that is routinely used is chain termination sequencing, in which modified nucleotides that terminate DNA strand elongation. In chain termination sequencing, a sequencing reaction is divided into four separate sequencing reactions, each containing all four of the standard deoxynucleotides, a radiolabeled nucleotide, a template strand, a divalent cation, and a DNA polymerase. To each of the four reactions, one of four dideoxynucleotides (ddATP, ddGTP, ddCTP, or ddTTP) are added. Dideoxynucleotides are chain-terminating nucleotides because they lack a 3′-OH group required for the formation of a phosphodiester bond between two nucleotides, thus terminating DNA strand extension and resulting in various DNA fragments of varying length.

The newly synthesized and labeled DNA fragments are heat denatured, and separated by size (with a resolution of just one nucleotide) by gel electrophoresis on a denaturing polyacrylamide-urea gel with each of the four reactions run in one of four individual lanes (lanes A, T, G, C); the DNA bands are then visualized by autoradiography or UV light, and the DNA sequence can be directly read off the X-ray film or gel image.

Dye-terminator sequencing is a variation of the chain termination methods in which each of the four chain terminator ddNTPs is labeled with a fluorescent dye that has a unique wavelengths of fluorescence and emission. This strategy circumvents the need for four separate reactions, since all four fluorescent signals can be run and read, e.g., in the same lane on a gel or in the same capillary in a capillary electrophoresis system.

The high demand for large-scale sequencing has driven the development of high-throughput sequencing technologies that parallelize the sequencing process, producing thousands or millions of sequences at once. High-throughput sequencing technologies can lower the cost of sequencing, e.g., target nucleic acids isolated using the methods and compositions described herein, beyond what is possible with standard dye-terminator or chain termination methods. Certain commercial high-throughput sequencing systems, e.g., those available from 454 Life Sciences, Illumina, Pacific Biosciences, and others, are based on multiplexed direct sequencing methods, e.g., “sequencing by synthesis” (SBS), in which each base position in a single-stranded DNA template is determined individually during the synthesis of a complementary strand.

The targeted resequencing of, e.g., nucleic acids isolated using capture probes, e.g., according to any one or combination of methods discussed herein, can be integrated with any of a variety of high-throughput DNA sequencing systems (reviewed in, e.g., Chan et al. (2005) “Advances in Sequencing Technology” (Review) Mutation Research 573: 13-40) to permit large-scale resequencing efforts of, e.g., complex genomes. See, e.g., Hodges et al. (2007) “Genome-wide in situ exon capture for selective resequencing.” Nat Genet 39: 1522-1527; Olson (2007) “Enrichment of super-sized resequencing targets from the human genome.” Nat Methods 4: 891-892; Porreca et al. (2007) “Multiplex amplification of large sets of human exons.” Nat Methods 4: 931-936.

One subset of commercial sequencing systems, e.g., those available from Affymetrix and Complete Genomics, Inc., rely on indirect methods of determining a DNA's sequence, e.g., sequencing by hybridization (SBH), in which a sequence of a DNA, e.g., a target nucleic acid, is assembled based on experimental data obtained from hybridization experiments performed to determine the oligonucleotide content of the target nucleic acid. See, e.g., Drmanac et al. (2002) “Sequencing by hybridization (SBH): advantages, achievements, and opportunities.” Adv Biochem Eng Biotechnol 77: 75-101. SBH typically employs an array comprising a known arrangement of short oligonucleotides of known sequence, e.g., oligonucleotides representing all possible sequences of a given length. An unknown sequence of, e.g., fluorescently labeled target DNA, is fragmented, and the resulting fragments are then hybridized to the oligonucleotide probes in the array. For example, a target nucleic acid isolated according to the methods of the invention, e.g., using probes provided by the invention, can be fluorescently labeled, fragmented and hybridized to such an array. Because the hybridization of a nucleic acid to a short complementary sequence can be sensitive to even single-base mismatches, the hybridization intensity of the labeled target nucleic acid fragments to individual probes in the array is computationally assessed to determine the sequences of the fragments. Additional computational approaches are then used to assemble the sequence fragments to determine the entire sequence of the target nucleic acid whose fragments were hybridized to the array.

Other commercial high-throughput sequencing systems, e.g., those available from 454 Life Sciences, Illumina, and Pacific Biosciences, are based on multiplexed direct sequencing methods, e.g., “sequencing by synthesis” (SBS), in which each base position in a single-stranded DNA template is determined individually during the synthesis of a complementary strand.

For example, pyrosequencing is a bioluminometric DNA sequencing technique in which the real-time release of the inorganic pyrophosphate (PPi) that is produced upon each successful incorporation of a nucleotide into a DNA is monitored (Nyren (2007) “The History of Pyrosequencing.” Methods Mol Biol 373: 1-14; Ronaghi (2001) “Pyrosequencing sheds light on DNA sequencing.” Genome Res 11: 3-11; and Wheeler et al. (2008) “The complete genome of an individual by massively parallel DNA sequencing.” Nature 452: 872-876). In pyrosequencing, PPi release begins an enzymatic cascade in which PPi is immediately converted to ATP by ATP sulfurylase. The ATP then fuels the luciferase-catalyzed oxidation luciferin, in which photons are emitted.

454 Sequencing, a technology available from 454 Life Sciences, is a massively-parallellized, multiplex pyrosequencing system that relies on fixing nebulized, adapter-ligated single-stranded DNA fragments to small DNA-capture beads. The single-stranded DNAs fixed to these beads is then amplified, e.g., via PCR. For example, target nucleic acids isolated using the methods and compositions described herein can be tethered to such beads and then amplified. Each target nucleic acid-bound bead can then be placed into a well on a proprietary PicoTiterPlate™, to which a mix of enzymes, including, e.g., DNA polymerase, ATP sulfurylase, and luciferase, has also been added. The PicoTiterPlat™ is then placed into a sequencing module, where dideoxyribonucleotides, e.g., A, C, G, and T, are washed in series over the PicoTiterPlate™. During the nucleotide flow, the copies of target nucleic acids that are attached to the beads are sequenced in parallel. If a nucleotide complementary to a target nucleic acid strand is flowed into a well of the PicoTiterPlate™, the polymerase extends the existing DNA strand by adding the nucleotide, releasing PPi and generating a light signal. The presence or absence of PPi, and, therefore, the incorporation or non-incorporation of each nucleotide washed over the PicoTiterPlate™, is ultimately assessed on the basis of whether or not photons are detected. There is a minimal time lapse between these events, and the conditions of the reaction are such that iterative addition of nucleotides and PPi detection are possible. Recently, 454 Sequencing technology was used to determine the complete sequence of an individual's genome at a cost of approximately $2,000,000 (Wheeler et al. (2008) “The complete genome of an individual by massively parallel DNA sequencing.” Nature 452: 872-876), a 5-fold reduction in costs compared to that of sequencing an individual's genome using Sanger dideoxy sequencing methods (Levy et al., (2007) “The Diploid Genome Sequence of an Individual Human.” PLoS Biol 5: e254).

Single molecule real-time sequencing (SMRT) is another massively parallel sequencing technology that can be compatible with the high-throughput resequencing of target nucleic acids isolated isolated from a sample, e.g., by using capture probes synthesized according to any of the methods described previously. Developed and commercialized by Pacific Biosciences, SMRT technology relies on arrays of multiplexed zero-mode waveguides (ZMWs) in which, e.g., thousands of sequencing reactions can take place simultaneously. The ZMW is a structure that creates an illuminated observation volume that is small enough to observe, e.g., the template-dependent synthesis of a single single-stranded DNA molecule, e.g., a single strand of a target nucleic acid isolated according to the methods and compositions provided by the inventon, by a single DNA polymerase (See, e.g., Eid et al (2008) “Real-Time DNA Sequencing from Single Polymerase Molecules.” Science 323: 133-138; Levene et al. (2003) “Zero Mode Waveguides for Single Molecule Analysis at High Concentrations,” Science 299: 682-686). When a DNA polymerase incorporates complementary, fluorescently labeled nucleotides into the DNA strand that is being synthesized, the enzyme holds each nucleotide within the detection volume for tens of milliseconds, e.g., orders of magnitude longer than the amount of time it takes an unincorporated nucleotide to diffuse in and out of the detection volume. During this time, the fluorophore emits fluorescent light whose color corresponds to the nucleotide base's identity. Then, as part of the nucleotide incorporation cycle, the polymerase cleaves the bond that previously held the fluorophore in place and the dye diffuses out of the detection volume. Following incorporation, the signal immediately returns to baseline and the process repeats. Target nucleic acids isolated as described herein can be deatured, optionally circularized, and distribted to the wells of a ZMW, and can thus be advantageously prepared for sequencing in a SMRT system.

In a preferred embodiment, target nucleic acids isolated from nucleic acid samples, e.g., by probes synthesized by methods provided by the invention, are sequenced using systems that include bridge amplification technologies, e.g., in which primers bound to a solid phase are used in the extension and amplification of solution phase target nucleic acid acids prior to SBS. (See, e.g., Mercier et al. (2005) “Solid Phase DNA Amplification: A Brownian Dynamics Study of Crowding Effects.” Biophysical Journal 89: 32-42; Bing et al. (1996) “Bridge Amplification: A Solid Phase PCR System for the Amplification and Detection of Allelic Differences in Single Copy Genes.” Proceedings of the Seventh International Symposium on Human Identification, Promega Corporation Madison, Wis.) Solexa sequencing, available from Illumina, is one such sequencing system.

Target nucleic acids can be prepared for sequencing, e.g., using the Solexa system, in the following manner. After the “capture” and enrichment of target nucleic acids, (see, e.g., FIGS. 6, 8 and the corresponding description above), unique adapters are attached to the ends of the target nucleic acids during sample preparation (see, e.g., FIG. 7 and the corresponding description above). Methods, described hereinbelow, by which the adapters are attached to the target nucleic acids are not particularly limiting. The target nucleic acids to which the adapters have been attached can then be amplified in a “bridged” amplification reaction on the surface of a flow cell. The flow cell surface is coated with single stranded oligonucleotides that correspond to the sequences of the adapters ligated to the target nucleic acids during sample preparation. Single-stranded, adapter-ligated fragments are bound to the surface of the flow cell and exposed to reagents for polymerase-based extension. Priming occurs as the free/distal end of a ligated fragment “bridges” to a complementary oligonucleotide on the surface, and during the annealing step, the extension product from one bound primer forms a second bridge strand to the other bound primer. Repeated denaturation and extension results in localized amplification of single molecules in millions of unique locations, creating clonal “clusters” across the flow cell surface.

The flow cell is then placed in a fluidics cassette within a sequencing module, where primers, DNA polymerase, and fluorescently-labeled, reversibly terminated nucleotides, e.g., A, C, G, and T, are added to permit the incorporation of a single nucleotide into each clonal DNA in each cluster. Each incorporation step is followed by the high-resolution imaging of the entire flow cell to identify the nucleotides that were incorporated at each cluster location on the flow cell. After the imaging step, a chemical step is performed to deblock the 3′ ends of the incorporated nucleotides to permit the subsequent incorporation of another nucleotide. Iterative cycles are performed to generate a series of images each representing a single base extension at a specific cluster. This system typically produces sequence reads of up to 20-50 nucleotides. Further details regarding this sequencing system are discussed in, e.g., Bennett et al. (2005) “Toward the 1,000 dollars human genome.” Pharmacogenomics 6: 373-382; Bennett (2004) “Solexa Ltd.” Pharmacogenomics 5: 433-438; and Bentley (2006) “Whole genome re-sequencing.” Curr Opin Genet Dev 16: 545-52.

Thus, target nucleic acids that have been isolated from a nucleic acid sample using the methods and compositions of the invention can be efficiently prepared for sequencing by a variety of high-throughput SBH and SBS platforms. Not only can such target nucleic acids be prepared for sequencing at low cost, the methods described herein for the purification of the target nucleic acids from a sample offer additional benefits over, e.g., PCR-based methods of preparing nucleic acids for resequencing. Capture probes, e.g., produced as described herein, when used to isolate target nucleic acids of interest can significantly reduce the time, effort, and expense necessitated by the parallel design, optimization, and execution of up to, e.g., thousands of individual PCR reactions that are typically performed to amplify, e.g., genes, transcripts, or genomic loci of interest for resequencing. For example, the amplification of, e.g., multiple genes or genomic loci, entails the parallel design, optimization, and execution of up to, e.g., thousands of individual PCR reactions, representing a substantial investment in time, effort, and money. In addition, preparing samples for resequencing via PCR can be technically challenging if enriching a locus that comprises the sequence of interest requires the amplification of nucleic acid fragments that are longer than a few hundred kilobases. Repetitive regions, which are typical of complex genomes, can be difficult to amplify using PCR. Furthermore, PCR can only be used to amplify fragments that are known to comprise the sequence(s) of interest, precluding the discovery of, e.g., additional genes, genomic loci, or the like, that also comprise such sequence(s). In contrast, the compositions and methods provided by the invention can be beneficially used to identify, e.g., novel genomic loci, genes, transcripts, and the like, that comprise a target subsequence of interest.

### Further Details Regarding Arrays

Compositions provided by the invention include arrays of transcribable nucleic acids that are tethered to a solid support (see, e.g., nucleic acid 100 and solid support 105 in FIG. 1 and the corresponding description above) from which, e.g., RNAs can be synthesized and nucleic acid capture probes can be produced. Such capture probes can subsequently be used to isolate desirable nucleic acids (e.g., “target nucleic acids”) comprising a subsequence of interest (e.g., “target subsequence”) from a population of nucleic acids, e.g., a population of shRNAs, a population of miRNAs, a population of mRNAs, a population of cDNAs, a population of fragments derived from a genomic DNA, total RNA derived from a cell, or the like. The nucleic acids tethered to the support (see, e.g., nucleic acid 100 in FIG. 1) generally comprise a single strand of a promoter sequence that is recognized by an RNA polymerase (see, e.g., promoter sequence 115). Tethered nucleic acids of the compositions also each include a selector subsequence of interest down stream of the promoter (see, e.g., selector subsequence 120 downstream of promoter 115 in FIG. 1) and, optionally, a first strand of unique restriction endonuclease recognition site (see restriction endonuclease restriction site 125). The tethered nucleic acids can optionally include constant regions (see, e.g., constant region 130 in FIG. 1), which can comprise processing features, e.g., one strand of a unique restriction endonuclease recognition site), which can facilitate the production of, e.g., a nucleic acid capture probe, as described in further detail above.

For example, a composition provided by the invention can comprise an array that includes a solid support to which, e.g., up to 1,000 nucleic acids, up to 10,000 nucleic acids, or, in some embodiments, up to 100,000 nucleic acids can be tethered, e.g., via their 5′ ends. Compositions provided by the invention include libraries, e.g., exon libraries, that can comprise one or more arrays of transcribable tethered nucleic acids, e.g., tethered nucleic acids from which nucleic acid capture probes can be produced, e.g., using methods described herein. The nucleic acids tethered to the one or more arrays can represent any number of unique selector subsequences, e.g., 1 unique selector subsequence per 10,000 tethered nucleic acids, 1 unique selector subsequence per 1,000 tethered nucleic acids, or even 1 unique selector subsequence per tethered nucleic acid.

Arrays of the invention can be manufactured in any of a variety of ways, depending on the number of nucleic acid probes they comprise, the materials from which the solid support is made, array component costs, customization requirements (e.g., for integration into existing systems), and the applications to which the arrays are put. Arrays can have as few as 1 nucleic acid type (e.g., from which one unique capture probe can be produced), and can also include up to about 500,000 or more nucleic acid types (e.g., from which 500,000 unique capture probes can be produced) arranged in micron-scale probe features, using current technology. Arrays can be arranged on solid supports (beads, plus planar surfaces), can be in liquid (e.g., in microtiter plates), or can be arranged on solid supports that are, themselves, arranged into physical or logical arrays in microtiter plates, or the like. The arrays can be arranged on a planar substrate, a bead or set of beads, a slide, a microscope slide, a micro-well plate, or a combination thereof.

In standard microarrays, nucleic acids can be bound to a solid surface by covalent attachment to a chemical matrix (e.g., via epoxy-silane, amino-silane, lysine, polyacrylamide or others). The solid surface can be, e.g., glass or other ceramic, polymer, or a silicon chip, commonly known as “gene chip”. Some microarray platforms, such as those used by Illumina, utilize microscopic beads, instead of the large solid supports (glass or treated silicon) used in traditional microarrays.

Accordingly, the type of solid support can vary in the methods, compositions, libraries and systems of the invention, based on the intended application. Solid support materials include, but are not limited to, glass, polyacrylamide, silica, controlled pore glass (CPG), polystyrene, polystyrene/latex, carboxyl modified teflon, nylon and nitrocellulose. The solid substrates can be biological, nonbiological, organic, inorganic, or a combination of any of these, existing as particles, strands, precipitates, gels, sheets, tubing, spheres, containers, capillaries, pads, slices, films, plates, slides, etc., depending upon the particular application. Other suitable solid substrate materials will be readily apparent to those of skill in the art.

Often, the surface of the solid substrate will contain reactive groups, such as carboxyl, amino, hydroxyl, thiol, or the like, e.g., for the attachment of nucleic acids (or other possible array components, such as proteins), etc. For example, in the present invention, nucleic acids are preferably attached to a glass slide or a magnetic bead. Surfaces on the solid substrate will sometimes, though not always, be composed of the same material as the substrate. Thus, the surface can be composed of any of a wide variety of materials, for example, polymers, plastics, resins, polysaccharides, silica or silica-based materials, carbon, metals, inorganic glasses, membranes, or any of the above-listed substrate materials. The surface may also be chemically modified or functionalized in such a way as to allow it to establish binding interactions with functional groups intrinsic to or specifically associated with the nucleic acids to be immobilized.

Arrays of the invention can be created by chip masking or tiling methods, e.g., using photoactivatable chemistry, can be spotted onto appropriate surfaces, e.g., using ink jets or pins, can be combinatorially produced e.g., as in bead-based approaches, or the like. In preferred embodiments to the invention, arrays of nucleic acids from which capture probes can be produced via standard column chemistries well known in the art. The oligos can then be captured onto beads, slides, or the like using any one of a variety of linking chemistries well known in the art. Many commercially available kits (e.g., from Invitrogen) can be used to capture oligos to, e.g., a bead, to create an array of the invention. (See Example 2.) Alternately, the oligos and/or arrays can be custom ordered from Aligent. Further details regarding coupling of nucleic acids to arrays, array formats, applications, and array analysis can be found, e.g., in: Kimmel and Oliver (eds) (2006) DNA Microarrays Part A: Array Platforms & Wet-Bench Protocols, Volume 410 (Methods in Enzymology) Academic Press; 1st edition ISBN-10: 0121828158; Kimmel and Oliver (2006) DNA Microarrays, Part B: Databases and Statistics, Volume 411 (Methods in Enzymology) Academic Press; 1st edition ISBN-10: 0121828166; Primrose and Twyman (2006) Principles of Gene Manipulation and Genomics Wiley-Blackwell, 7th edition ISBN-10: 1405135441; Gibson and Muse (2004) A Primer of Genome Science, 2nd Edition Sinauer Associates; 2nd edition ISBN-10: 0878932321; Lausted et al. (2004) POSaM: a fast, flexible, open-source, inkjet oligonucleotide synthesizer and microarrayer Genome Biol 5: R58.Published online 2004 Jul. 27. doi: 10.1186/gb-2004-5-8-r58; Draghici (2003) Data Analysis Tools for DNA Microarrays Chapman & Hall/CRC; ISBN-10: 1584883154; Stekel (2003) Microarray Bioinformatics Cambridge University Press; 1st edition # ISBN-10: 052152587X; Baldi et al. (2002) DNA Microarrays and Gene Expression: From Experiments to Data Analysis and Modeling Cambridge University Press; 1st edition ISBN-10: 0521800226; and DNA Microarrays: Gene Expression Applications (2001) B. R. Jordan (Editor) Springer; 1st edition ISBN-10: 3540415076.

Arrays of the invention can optionally be arranged in physical gridded arrangements of array members, e.g., as is typical for a gene chip, or a bead array set in microtiter trays. However, the array can also take on non-traditional formats, e.g., a logical array can be, e.g., a virtual arrangement of the member set in a computer system, or e.g., an arrangement of set elements produced by performing a specified physical manipulation on one or more set element or components of set elements. For example, a logical array can be described in which set members (or components that can be combined to produce set members) can be transported or manipulated to produce the set. Further details on these general approaches are found in the references noted above.

Arrays can also be duplicated, e.g., to increase production of transcripts (e.g., RNA 150 in FIG. 1), or for the commercial sale of arrays. A “duplicate” or “copy” array is an array that can at least partially be corresponded to a parental array. In simplest form, this correspondence takes the form of simply replicating all or part of the parental array, e.g., by taking aliquots of material from each position in the parental array (or otherwise reproducing the array, e.g., by duplicate synthesis of nucleic acids on solid supports). However, any method that results in the ability to correspond members of the duplicate array to the parental array can be used for array duplication, including the use of complex storage algorithms, partially or purely in silico arrays, and pooling approaches which partially combine some elements of the parental array into single locations (physical or virtual) in the duplicate array. The duplicate or copy array duplicates some or all components of a parental array. For example, an array of reaction mixtures might include RNAs fixed to solid supports along with other relevant components, such as transcription reagents at sites in the array.

### Further Details Regarding Systems

The methods and compositions provided by the invention can advantageously be integrated with systems which can, e.g., automate the production of nucleic acid capture probes and/or the resequencing target nucleic acids retrieved from a population of nucleic acids using the capture probes. Systems of the invention can include one or more modules, e.g., that automate a method herein, e.g., for high-throughput applications. Such systems can include arrays, fluid handling elements and controllers that move reaction components, e.g., nucleotide mixes, enzymes, oligonucleotide primers, etc. into contact with one another, sequencing apparatuses that utilize nucleic acids produced by the methods herein in various sequencing reactions, e.g., as described above, signal detectors, system software/instructions, and the like.

In general, arrays, which are discussed in further detail above, can be used with fluid handling elements that move enzymes, primers, or the like into contact with the arrays of the invention. The format of the fluid handling elements will depend on the format of the array. For example, arrays that are laid down on a solid surface, e.g., in a typical gene chip format can utilize flow controllers that deliver fluids comprising the enzymes, primers, or the like to the appropriate regions of the solid surface where a reaction is desired. Similarly, a variety of automated flow controllers exist for the deliver of fluids to microtiter trays, facilitating construction of an overall system that utilizes one or more arrays of the invention.

In general, materials, such as nucleotides, restriction enzymes, polymerases, reverse transcrtiptases, oligonucleotide primers, and the like, can be delivered to an array (see, e.g., array 108 in FIG. 1A and corresponding description) by methods that are generally used to deliver analyte molecules to an array, e.g., an array of tethered nucleotide from which capture probes can be produced (see, e.g., FIG. 1 and corresponding description). For example, delivery methods can include suspending the reagents in a fluid and flowing the resulting suspension onto an array or into wells of an array. This can include simply pipetting the relevant suspension onto one or more regions of the array, or can include more active flow methods, such as electrodirection or pressure-based fluid flow. In one useful embodiment, reagents are flowed into selected regions of the array. This can be accomplished by masking techniques (applying a mask to direct fluid flow), or by active flow methods such as electrodirection or pressure based fluid flow, as described above, including by ink-jet printing methods. Ink jet and other delivery methods for delivering nucleic acids and related reagents to arrays is found, e.g., in Lausted et al. (2004) POSaM: a fast, flexible, open-source, inkjet oligonucleotide synthesizer and microarrayer Genome Biol 5: R58.Published online 2004 Jul. 27. doi: 10.1186/gb-2004-5-8-r58; Kimmel and Oliver (Eds) (2006) DNA Microarrays Part A: Array Platforms & Wet-Bench Protocols, Volume 410 (Methods in Enzymology) ISBN-10: 0121828158; Lee (2002) Microdrop Generation (Nano- and Microscience, Engineering, Technology and Medicine) CRC Press ISBN-10: 084931559X; and Heller (2002) “DNA MICROARRAY TECHNOLOGY: Devices, Systems, and Applications” Annual Review of Biomedical Engineering 4: 129-153. Regions of an array can also be selective targets of delivery simply by pipetting the relevant suspension into the correct region of the array.

Furthermore, several “off the shelf” fluid handling stations for performing such transfers are commercially available, including e.g., the Zymark Zymate, Twister Microplate Handler, Sciclone family of liquid handling systems, and the Zephyr Liquid Handler, all from Caliper Technologies (Hopkinton, Mass.). Chemical inkjet printers for reagent delivery are available from a variety of sources, such as Shimadzu Biotech (Japan, and Columbia, Md.).

In general, these and other available fluid handlers utilize automatic pipettors, piezo electric elements, or the like, e.g., in conjunction with the robotics for plate movement. In an alternate embodiment, fluid handling for making capture probes is performed in microchips, e.g., involving transfer of materials from microwell plates or other wells through microchannels on the chips to destination sites (microchannel regions, wells, chambers or the like). Commercially available microfluidic systems include those from Hewlett-Packard/Agilent Technologies (e.g., the HP2100 bioanalyzer) and the Caliper High Throughput Screening Systems. The Caliper High Throughput Screening Systems, such as the LabChip 3000™, provide an interface between standard library formats and chip technologies. Furthermore, the patent and technical literature includes examples of microfluidic systems that can interface directly with microwell plates for fluid handling, e.g., fluids in which reagents (enzymes, oligos, nucleotides) for the production of capture probes have been suspended or fluids in which capture probes have been suspended in preparation for purifying and isolating target nucleic acids of interest from a nucleic acid sample.

In one embodiment, a system of the invention can also integrate a processing module, such as a thermocycling apparatus to perform enzymatic reactions. Such a module can be integral with the fluid handling apparatus (e.g., as in “on chip” enzymatic reactions, performed using the systems described above), or separate from it, e.g., where the fluid handling systems deliver the appropriate enzymatic reaction components to an incubation station, thermocycler, or the like. In the present invention, a thermocycler can be used in a variety of step in producing capture probes, e.g., to anneal oligos to tethered nucleic acids in preparation for transcription (FIG. 1) and to perform the subsequent transcription reaction; to anneal oligos to RNAs in preparation for reverse transcription (FIGS. 2 and 3) and to perform the subsequent reverse transcription reaction; in preparing the captured target nucleic acids for sequencing (FIGS. 7 and 8) etc A myriad of automated or automatable processing module elements, such as thermocyclers, incubators, or the like are commercially available.

An overall system provided by the invention can also comprise a sequencing apparatus, e.g., any of the currently available super high throughput sequencing systems, which are discussed in further detail above. Such a system can be advantageously used to sequence “captured” nucleic acids, e.g., target nucleic acids that have been isolated using the methods and compositions described herein. One such system, available from 454 Life Sciences, was used to determine an individual's complete genome sequence (Wheeler et al. (2008) “The complete genome of an individual by massively parallel DNA sequencing.” Nature 452: 872-876). In general, the fluid handling elements can incorporate sequencing module components, or can deliver products of the processing modules to a sequencing module/station. Sequencing stations are commercially available, e.g., from Illumina (San Diego, Calif.), see, e.g., the 2008 Illumina Product Guide, and Applera/Applied Biosystems (Foster, Calif.), e.g., using capillary electrophoresis and cycle sequencing chemistries, and/or the SOLiD™ system. For example, the Illumina genome analyzer station can be integrated with available fluid handling equipment to provide templates from the methods of the invention to the sequencing station. For example, whole genome re-sequencing stations (e.g., Bently (2006) Whole-Genome Re-sequencing Curr Opin Genet Dev 16: 545-52) can utilize template nucleic acids produced according to the methods herein. Fluid handling systems can be used to flow/transfer templates for sequencing from the process modules to the sequencing system.

Systems of the invention can optionally include modules that provide for detection or tracking of sequencing reaction products, e.g., generated during the sequencing of target nucleic acids that have been isolated from a nucleic acid sample, e.g., according to the methods of the invention, e.g., using capture probes of the invention. Such modules can be particularly useful in, e.g., a bioluminometric DNA sequencing systems, e.g., available from 454 Life Sciences and others. Detectors can include spectrophotometers, CCD arrays, microscopes, cameras, or the like. Optical labeling is particularly useful because of the sensitivity and ease of detection of these labels, as well as their relative handling safety, and the ease of integration with available detection systems (e.g., using microscopes, cameras, photomultipliers, CCD arrays and/or combinations thereof). High-throughput analysis systems using optical labels include DNA sequencers, array readout systems, and the like. For a brief overview of fluorescent products and technologies see, e.g., Sullivan (ed) (2007) Fluorescent Proteins, Volume 85, Second Edition (Methods in Cell Biology) (Methods in Cell Biology) ISBN-10: 0123725585; Hof et al. (eds) (2005) Fluorescence Spectroscopy in Biology: Advanced Methods and their Applications to Membranes, Proteins, DNA, and Cells (Springer Series on Fluorescence) ISBN-10: 354022338X; Haughland (2005) Handbook of Fluorescent Probes and Research Products, 10th Edition (Invitrogen, Inc./Molecular Probes); BioProbes Handbook, (2002) from Molecular Probes, Inc.; and Valeur (2001) Molecular Fluorescence: Principles and Applications Wiley ISBN-10: 352729919X.

System software, e.g., instructions running on a computer can be used to track and inventory reactants or products, and/or for controlling robotics/fluid handlers to achieve transfer between system stations/modules. The overall system can optionally be integrated into a single apparatus, or can consist of multiple apparatus with overall system software/instructions providing an operable linkage between modules.

Systems of the invention can include one or more output devices, such as a printer and/or a monitor to display results, and the like

In certain embodiments of the invention, specific adaptor sequences, e.g., nucleic acid adaptors available from commercial sequencing systems such as Illumina, can be hybridized or ligated to the ends of the captured DNAs and/or captured RNAs to facilitate resequencing of these target nucleic acids, e.g., by an sequencing system available from Illumina.

### Additional Details Regarding Downstream Applications in Which Capture Probes can be Used

Nucleic acid capture probes, e.g., produced according to the methods described herein, can be used in a variety of applications which entail the enrichment and resequencing of nucleic acids comprising a target subsequence of interest from a sample population of nucleic acids (See FIGS. 6, 8, and the corresponding description above). The application(s) in which a particular capture probe, or set of capture probes, can be used is generally delimited by the probes' encoded selector subsequences. Accordingly, the number of unique target nucleic acids that can be isolated from a nucleic acid sample can be contingent upon the source from which the sample was derived, e.g., organism, tissue, germline cell, somatic cell, cell type, organelle, the developmental stage of the source at the time the sample was prepared, the disease state of the source, and/or environmental influences on the source. Similarly, the set of target nucleic acids enriched by the capture probes can be restricted by the type of nucleic acid, e.g., genomic DNA, cDNA, mRNA, miRNA, DNA encoding introns, or kinds of nucleic acids, present in the sample. Nevertheless, the nucleotide sequence of the selector subsequence of a capture probe is not particularly limiting, and the nucleic acid samples that can be interrogated by, e.g., a capture probe or a set of capture probes, can comprise any kind of nucleic acid derived from any source. Methods of nucleic acid sample preparation are described in further detail below.

The number of unique capture probes, e.g., capture probes comprising unique selector subsequences, that can be used to simultaneously interrogate a nucleic acid sample is not limiting. Consequently, capture probes, e.g., synthesized according to the methods of the invention, are well suited for use in applications that entail the parallel enrichment of a plurality of unique target subsequences present in a plurality or target nucleic acids, e.g., up to 10 target subsequences, up to 100 target subsequences, or up to 500 target subsequences, from a sample. A population of capture probes in which at least 10,000 unique selector subsequences, at least 100,000 unique selector subsequences, or at least 1,000,000 unique selector subsequences are represented can be simultaneously produced from a library provided by the invention and used in a parallel interrogation of a nucleic acid sample.

One application in which capture probes, e.g., produced according to methods described herein, can be of beneficial use is in the targeted resequencing of one or more loci of interest in a genome, e.g., a genomic locus associated with a disease state, to identify a mutation and make a diagnosis. For example, a capture probe that comprises a selector subsequence complementary to a target subsequence present in, e.g., a tumor suppressor gene, can be used to interrogate a sample comprising genomic DNA derived from, e.g., a tissue biopsied from a patient. The target nucleic acid(s) isolated by the capture probe can be resequenced, e.g., using a high-throughput sequencing system, and the sequence(s) can be compared to a reference genome to, e.g., diagnose the patient's disease state or determine patient's susceptibility to the disease. Because the sequences of the target nucleic acids will have been determined, the nature the mutations, e.g., deletion, insertion, frameshift, etc., at a locus in an individual's genome can be ascertained, as can the frequency with which a specific mutation at that locus arises amongst patients with the disease.

In a similar aspect, capture probes produced by the methods of the invention can be used in massively parallel interrogations of, e.g., genomic DNA samples derived from numerous subjects to, e.g., detect rare alleles or determine the frequency with which characteristic single nucleotide polymorphism (SNP) profiles or haplotypes correlate with disease susceptibilities in given population. One method by which SNP profiles can be determined is by sequencing and comparing numerous individuals' entire genomes. The complete genomes of two individuals have recently been sequenced. One genome was sequenced using Sanger dideoxy technology (Levy et al., (2007) “The Diploid Genome Sequence of an Individual Human.” PLoS Biol 5: e254) at a cost of $10,000,000, and the other was sequenced using a high-throughput sequencing system available from 454 Life Sciences (Wheeler et al. (2008) “The complete genome of an individual by massively parallel DNA sequencing.” Nature 452: 872-876) at a cost of $2,000,000. Though the costs of sequencing a second human genome were reduced by a factor of 5 relative to the first, using even recently developed high-throughput sequencing technologies can be too costly and laborious to sequence the complete genomes of more than a small number of individuals. Resequencing, or the targeted sequencing of one or more segments, regions, or loci of interest of nucleic acid sample of interest, can be a particularly useful, cost-effective method of detecting mutations associated with various complex human diseases, including cancer, heart disease, and others. However, one of the major challenges of resequencing is the efficient isolation of the target nucleic acids to be sequenced.

Typically, PCR has been used to amplify regions of interest from, e.g., a nucleic acid or population of nucleic acids extracted from a biological sample in preparation for resequencing. However, using PCR to amplify regions of interest in, e.g., a genome, a population of cDNAs, or a population of RNAs, for resequencing, can limit the length of the sequence that is amplified. Repetitive regions, which are typical of complex genomes, can be difficult to amplify using PCR. Furthermore, multiplexing PCR for the enrichment of, e.g., several thousand regions of interest in a nucleic acid sample, can be both expensive and labor-intensive. Isolating target nucleic acids, from a sample population of nucleic acids, e.g., using capture probes produced by the invention to perform the methods provided be the invention, can be a cost-effective, labor-saving alternative to the parallel design, optimization, and execution of up to, e.g., thousands of individual PCR reactions.

Resequencing can advance the study of, e.g., the relationship between sequence variation and normal or disease phenotypes. Discovering the genetic profiles that are associated with particular diseases can also lead to the identification of new therapeutic targets. Accordingly, sets of capture probes, e.g., produced according to the methods described herein, can be used to interrogate individuals' genomes for SNP profiles which are associated with, e.g., abnormal drug metabolism, to inform and personalize a patient's therapeutic treatment, e.g., in order to avoid negative consequences associated with the use of a given medication.

CpG island DNA methylation plays an important role in regulating gene expression in, e.g., development and carcinogenesis. Capture probes produced by the methods of the invention can be used to interrogate genomic DNA samples for the methylation state at a particular locus, e.g., the promoter from which a tumor suppressor gene or an oncogene is transcribed. In short, a capture probe comprising a selector subsequence complementary to that of a promoter of interest can be used to isolate a target nucleic acid from, e.g., a genomic DNA sample that has been treated with bisufite. Bisulfite converts unmethylated cytosine residues to uracil, but leaves methylated cytosine residues, e.g., 5-methylcytosine residues, unaffected. Thus, the resequencing of, e.g., 20-30 nucleotides in the subsequences of interest of captured target nucleic acids, e.g., that have been retreived from samples that have been treated with bisulfite, can yield high-resolution information about the methylation status of the segment of genomic DNA that was isolated. This information can subsequently be used to diagnose or treat, e.g., cancer or other disease states that can result from changes in the transcriptional activity of a gene.

Capture probes can also be used in applications wherein populations of RNAs are interrogated, e.g., to isolate an RNAs that comprise a sequence of interest. In one application, capture probes that are designed to include a selector subsequence that can be used to interrogate a sample of RNAs to identify miRNA processing intermediates. MicroRNAs (miRNAs) are an abundant class of small single-stranded non-coding RNAs (19-30 nucleotides long) that serve widespread functions in post-transcriptional gene silencing (Zhang (2008) “MicroRNomics: a newly emerging approach for disease biology.” Physiol Genomics 33: 139-47). miRNAs hac been shown to play a role in the regulation of gene expression, affecting a wide variety of cellular functions including development, proliferation, differentiation, and apoptosis (Shyu (2008) “Messenger RNA regulation: to translate or to degrade.” EMBO J 27: 471-81). Identifying post-transcriptional regulators of miRNA processing can be useful in determining, e.g., whether differentially processed precursor miRNAs lead to tissue-specific or temporal miRNA expression or whether miRNA precursors play roles in the cell independent of the functions of the mature miRNAs that are then produced.

A schematic diagram depicting a method of using a capture probe to identify miRNA processing intermediates is shown in FIG. 12. Capture probe 1200, which comprises a selector subsequence that is complementary to a target subsequence found in a mature miRNA can be used to interrogate a population of RNAs to identify the relative levels of each miRNA precursor, e.g., a pri-miRNA, a pre-miRNA, etc., in a sample of RNA obtained from, e.g., a tissue. For example, capture probe 1200 can be used to isolate pri-miRNA 1205 from a sample, and resequencing of the captured pri-mRNA would produce sequence read 1210. Similarly, capture probe 1200 can be used to isolate pre-miRNA 1215 and/or mature miRNA 1225, which, when resequenced, would produce sequence reads 1220 and 1230, respectively. As described above, in certain embodiments of the invention, a sequence read can comprise 20-30 nucleotides.

A population of mRNAs can be interrogated by a capture probe to identify transcripts of the same gene that have unique 3′UTR/polyA sites. This application schematically depicted in FIG. 10. Capture probe 1000 comprises a selector subsequence that is complementary to an internal target subsequence in a gene of interest and can be used to retrieve all the mRNA species or cDNA species, e.g., mRNA or cDNA 1020 and mRNA or cDNA 1025 from a sample that comprise that target subsequence. Resequencing the captured target mRNA species to produce, e.g., sequence read 1030, from RNA or cDNA 1020, and sequence read 1035, from mRNA or cDNA 1025, permits the discovery of these alternate transcript types. Such results could be verified using, e.g., capture probe 1010, which comprises a selector subsequence that is downstream of selector subsequence in capture probe 1000, to capture mRNAs/cDNAs 1020 and 1025 to produce sequence reads 1040 and 1045, respectively, e.g., sequence reads of about 20-30 nucleotides.

In a similar aspect, capture probes can be designed to interrogate a population of RNAs to identify transcripts of the same gene that were transcribed from alternate promoters. An example of such an interrogation experiment is schematically depicted in FIG. 11. Capture probe 1100 comprises a selector subsequence complementary to a target subsequence at, e.g., the 5′ end of a gene of interest, can be used to retrieve those mRNAs or cDNAs from a sample, e.g., mRNAs or cDNAs 1110 and 1115, that encode that target subsequence. Resequencing of the purified target nucleic acids can produce sequence reads, e.g., sequence reads 1120 and 1125, which verify that the gene of interest is transcribed from, e.g., more than one promoter.

Capture probes synthesized by a method provided by the invention can be used to interrogate a library of mRNAs or cDNAs to discover novel and/or alternative splice isoforms of a gene of interest (see, e.g., the schematic depiction in FIG. 9.) Alternative splice isoforms are generated when the exons of a pre-RNA are reconnected in any one of a variety of combinations during RNA splicing. Accordingly, alternative splice isoforms of a gene will each typically comprise a unique set of exon-exon junctions, e.g., sites where two exons abut one another, wherein the exons were previously separated by intervening RNA. For example, capture probe 900, which comprises a selector subsequence complementary to, e.g., a target subsequence at the 3′ end of a first exon in a gene of interest, can be used to retrieve those mRNAs or cDNAs, e.g., mRNAs or cDNAs 905 and 910, that comprise the exon target subsequence. The selector sequence of capture probe 900 comprises one and only one exon subsequence. The target nucleic acids can be resequenced to produce sequence reads, e.g., sequence reads 915 and 920, which indicate that the exon directly downstream of Exon1 in mRNA or cDNA 905 and the exon directly downstream of Exon 1 in mRNA or cDNA 910 are different. By determining the sequences of the exon-exon junctions in mRNA or cDNA 905 and mRNA or cDNA 910, it can be shown that the gene from which mRNA or cDNA 905 and mRNA or cDNA 910 are derived can be processed into alternate splice isoforms.

Computer-implemented techniques, such as those described in U.S. Pat. No. 7,340,349, by Bingham et al., use mathematical algorithms to determine the expression levels of splice isoforms of a gene in, e.g., a nucleic acid sample that has been hybridized to a microarray comprising exon-exon junction probes. However, the interpretation of results derived from exon-exon junction-based arrays can be complicated by the fact that each exon-exon junction probe can cross-hybridize to transcripts that comprises only one of the two exon subsequences present in the probe. Furthermore, exon-exon junction probes cannot be used to detect novel or cryptic splice sites. By using the capture probes to the invention to identify splice isoforms of a gene of interest, such experimental challenges can be avoided.

RNA samples can also be interrogated, e.g., by capture probes produced by the invention, to compare expression levels of, e.g., transcripts of interest from within a sample or transcripts of interest derived from two or more different samples, e.g., derived from two or more patients, two or more tissues, two or more developmental states of the same tissue, two or more tissues that have been exposed to different treatments, and the like. Transcript expression levels can be quantified, e.g., by a detection module in a system, by monitoring the number of times a particular sequence of a captured nucleic acid is “read” by a high-throughput sequencing system. Sets of capture probes can be used to simultaneously interrogate a sample of, e.g., mRNAs or cDNAs, to determine a gene expression profile. For example, resequencing target nucleic acids, e.g., isolated using the methods and compositions of the invention, can be advantagously used to analyze, e.g., stem cell pluropotency. Capture probes comprising selector sequences that correspond to validated gene expression markers can be used to to characterize, e.g., mouse or human embryonic stem (ES) cell identity and assess phenotypic variations between embryonic stem cell isolates.

In many of the examples detailed above, the methods described herein, e.g., to isolate target nucleic acids from a population of nucleic acids, can be used to identify, e.g., novel transcripts, genes, genomic loci, and the like, that would otherwise remain undetected by current techniques, e.g., wherein PCR is used to amplify fragment of interest from a nucleic acids sample.

### Additional Details Regarding Molecular Techniques

Preparing Nucleic Acid Samples

Capture probes, e.g., produced by the methods described herein, can be used to isolate and purify one or more nucleic acids that comprise target subsequences of interest from a nucleic acid sample. The source, e.g., organism, cell, tissue, etc., from which a nucleic acid sample is derived is not particularly limiting. One of skill in the art will also recognize that a nucleic acid sample can comprise, e.g., a population of shRNAs, a population of miRNAs, a population of mRNAs, a population of cDNAs, a population of fragments derived from a genomic DNA, total RNA derived from a cell, and/or the like.

For example, genomic DNA can be prepared, e.g., for capture experiments, from any source by three steps: cell lysis, deproteinization and recovery of DNA. These steps are adapted to the demands of the application, the requested yield, purity and molecular weight of the DNA, and the amount and history of the source. Further details regarding the isolation of genomic DNA can be found in Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, New York, 2008 (“Sambrook”); Current Protocols in Molecular Biology, F. M. Ausubel et al., eds., Current Protocols, a joint venture between Greene Publishing Associates, Inc. and John Wiley & Sons, Inc (“Ausubel”); Kaufman et al. (2003) Handbook of Molecular and Cellular Methods in Biology and Medicine Second Edition Ceske (ed) CRC Press (Kaufman); and The Nucleic Acid Protocols Handbook Ralph Rapley (ed) (2000) Cold Spring Harbor, Humana Press Inc (Rapley). In addition, many kits are commercially available for the purification of genomic DNA from cells, including Wizard™ Genomic DNA Purification Kit, available from Promega; Aqua Pure™ Genomic DNA Isolation Kit, available from BioRad; Easy-DNA™ Kit, available from Invitrogen; and DnEasy™ Tissue Kit, which is available from Qiagen.

RNAs or mRNAs can typically be isolated from almost any source using protocols and methods described in, e.g., Sambrook and Ausubel. The yield and quality of the isolated RNA can depend on, e.g., how a tissue is stored prior to RNA extraction, the means by which the tissue is disrupted during RNA extraction, or on the type of tissue from which the RNA is extracted. RNA isolation protocols can be optimized accordingly. Kits for the preparation of total RNA from cells are available from, e.g., Agilent, Qiagen, Sigma-Aldrich, and others. Many mRNA isolation kits are commercially available, e.g., the mRNA-ONLY™ Prokaryotic mRNA Isolation Kit and the mRNA-ONLY™ Eukaryotic mRNA Isolation Kit (Epicentre Biotechnologies), the FastTrack 2.0 mRNA Isolation Kit (Invitrogen), and the Easy-mRNA Kit (BioChain). In addition, mRNA from various sources, e.g., bovine, mouse, and human, and tissues, e.g., brain, blood, and heart, is commercially available from, e.g., BioChain (Hayward, Calif.), Ambion (Austin, Tex.), and Clontech (Mountainview, Calif.).

Once the purified mRNA is recovered, reverse transcriptase can be used to generate cDNAs from the mRNA templates. Methods and protocols for the production of cDNA from mRNAs, e.g., harvested from prokaryotes as well as eukaryotes, are elaborated in cDNA Library Protocols, I. G. Cowell, et al., eds., Humana Press, New Jersey, 1997, Sambrook and Ausubel. In addition, many kits are commercially available for the preparation of cDNA, including the Cells-to-cDNA™ II Kit (Ambion), the RETROscript™ Kit (Ambion), the CloneMiner™ cDNA Library Construction Kit (Invitrogen), and the Universal RiboClone® cDNA Synthesis System (Promega). Many companies, e.g., Agencourt Bioscience and Clontech, offer cDNA synthesis services.

Generating Nucleic Acid Fragments

The interrogation methods described elsewhere herein can entail the shearing the sample nucleic acids prior to hybridization with the capture probe. There exist a plethora of ways of generating nucleic acid fragments from a genomic DNA, a cDNA, an mRNA, or the like. These include, but are not limited to, mechanical methods, such as sonication, mechanical shearing, nebulization, hydroshearing, and the like; enzymatic methods, such as exonuclease digestion, restriction endonuclease digestion, and the like; and electrochemical cleavage. These methods are further described in Sambrook and Ausubel.

Transcribing Nucleic Acids

The compositions provided by the invention include arrays that comprise solid supports to which transcribable nucleic acids are tethered, e.g., by their 5′ ends (see, e.g., FIG. 1 and the corresponding description.) The nucleic acids of the composition comprise one strand of a promoter sequence which is capable of being recognized and transcribed by an RNA polymerase under conditions wherein the promoter sequence is double stranded, e.g., sufficiently double stranded. For example, the promoter sequence is double-stranded, e.g., sufficiently double-stranded, when an oligonucleotide comprising a complementary sequence anneals to a the promoter subsequence of a nucleic acid described above in such a manner as to permit an RNA polymerase to initiate transcription. In the absence of such a double-stranded promoter sequence, an RNA polymerase cannot typically adopt the transcription-competent “open complex” conformation that permits subsequent promoter clearance. The minimal promoter sequence which, when double-stranded, comprises all the elements that are necessary and sufficient for recognition by an RNA polymerase can vary, depending of the origin of the RNA polymerase, e.g., prokaryote, yeast, mammal, etc. In preferred compositions of the invention and in preferred methods of synthesizing RNAs, the nucleic acids comprise the single-stranded T7 promoter sequence TAATACGACTCACTATA (SEQ ID NO: 1), the single-stranded SP6 promoter sequence ATTTAGGTGACACTATA (SEQ ID NO: 2), and/or the single-stranded T3 promoter sequence AATTAACCCTCACTAAA (SEQ ID NO: 3).

In vitro transcription can proceed in a reaction that comprises a purified linear DNA template containing a sufficiently double-stranded promoter, e.g., the tethered nucleic acids described above, ribonucleotide triphosphates, a buffer system that includes DTT and Mg⁺⁺ ions, and an RNA polymerase that can recognize the promoter. The exact conditions used in the transcription reaction can vary depending on the preferred yield of RNA that is to be produced. The polymerases that are most preferably used in the methods provided herein include a T7 RNA polymerase, a T3 RNA polymerase, and an SP6 RNA polymerase. Further details regarding performing transcription reaction, optimizing in vitro transcription reactions, and harvesting RNAs produced in an in vitro transcription reaction are elaborated in “Sambrook”, in “Ausubel”, and in Grandi, Guido (2007) In Vitro Transcription and Translation Protocols, Volume 375 (Methods in Molecular Biology) Humana Press, Inc.; 2nd Edition ISBN: 9781588295583.

Attaching Tags to Nucleic Acids

As described elsewhere herein, nucleic acid tags can comprise any of a plethora of ligands, such as high-affinity DNA-binding proteins; modified nucleotides, such as methylated, biotinylated, or fluorinated nucleotides; and nucleotide analogs, such as dye-labeled nucleotides, non-hydrolysable nucleotides, or nucleotides comprising heavy atoms. Such reagents are widely available from a variety of vendors, including Perkin Elmer, Jena Bioscience and Sigma-Aldrich. Nucleic acid tags can also include oligonucleotides that comprise specific sequences, such as restriction sites, cis regulatory sites, oligonucleotide hybridization sites, protein binding sites, and the like. Such oligonucleotide tags can be custom synthesized by commercial suppliers such as Operon (Huntsville, Ala.), IDT (Coralville, Iowa) and Bioneer (Alameda, Calif.). The methods that can be used to join tags to nucleic acids of interest include chemical linkage, ligation, and extension of a primer by, e.g., a DNA polymerase or a reverse transcriptase. Further details regarding nucleic acid tags and the methods by which they are attached to nucleic acids of interest are elaborated in Sambrook and Ausubel.

DNA can be coupled to a very wide variety of tags using a wide variety of available technology. A wide variety of tags are useful for isolating, detecting and manipulating a DNA of interest in the methods herein. In one convenient application, a biotinylated phosphoramidite (or other labeled phosphoramidite) can be added directly to the 5′ end of an oligonucleotide during chemical synthesis, e.g., in an automated DNA/oligonucleotide synthesizer. Many labeled phosphoramidites are commercially available for this application, e.g., from Beckman-Coulter (Fullerton, Calif.), Invitrogen (Molecular Probes) (Carlsbad, Calif.) and many others. Phosphoramidite labeling results in precise and efficient labeling of a DNA with a tag of interest. Enzymatic DNA label in can also be used to incorporate, e.g., a biotinylated-, fluorescent- or other hapten-labeled deoxynucleotide triphosphate (dNTP) into an existing DNA substrate, e.g., using a DNA polymerase kinase, or terminal transferase.

In a number of aspects of the invention, end-labeling is preferred, so that the label can be conveniently removed in downstream processing steps. Other available labels/tags include any of a variety of polymers (PEG, and many others), nanoparticles (e.g., comprising magnetic materials, gold, or other metals e.g., using thiol chemistries), quantum dots, nanowires (e.g., CdTe—Au—CdTe nanowires), and the like. See also, Zhou et al. (2008) “A compact functional quantum Dot-DNA conjugate: preparation, hybridization, and specific label-free DNA detection” Langmuir 24: 1659-1664; Wang and Ozkan (2008) “Multisegment nanowire sensors for the detection of DNA molecules” Nano Lett 8: 398-404; Dias and Lindman (eds) (2008) DNA Interactions with Polymers and Surfactants Wiley-Interscience ISBN-10: 0470258187; Hosokawa et al. (2007) Nanoparticle Technology Handbook Elsevier Science ISBN-10: 044453122X; Kimmel and Oliver (2006) DNA Microarrays Part A: Array Platforms & Wet-Bench Protocols, Volume 410 (Methods in Enzymology) Academic Press; 1st edition ISBN-10: 0121828158; Csaki et al. (2002) “Gold nanoparticles as novel label for DNA diagnostics” Expert Review of Molecular Diagnostics 2: 187-193; and Day (1991) “Immobilization of polynucleotides on magnetic particles: Factors influencing hybridization efficiency” Biochem J 278(Pt 3): 735-740. One of skill will readily appreciate that appropriate technologies are available to use such tags for labeling, manipulating DNAs of interest and the like, e.g., by using the appropriate tag binding agent, applying a magnetic field, etc., as appropriate for the tag.

In some embodiments of the methods of producing a capture probe, a ligation reaction can be performed to attach, e.g., a polyA tail or a ribonucleotide sequence comprising a primer hybridization site, to an RNA (see, e.g., FIG. 3). Ligation is a method by which DNAs, RNAs, or DNAs and RNAs are joined with a covalent bond. Ligations are performed by incubating the nucleic acid fragments to be joined in the presence of buffer, rATP, and a ligase enzyme capable of catalyzing the ligation reaction of interest. Further details regarding these techniques can be found in Sambrook and Ausubel. Furthermore, a plethora of enzymes, each capable of catalyzing a unique type of ligation reaction, are commercially available. For example, CircLigase™, from Epicentre Biotechnologies, is capable of catalyzing the intramolecular ligation of single-stranded DNA fragments; T4 RNA ligase 1, available from New England Biosciences, is capable of ligating single-stranded RNAs to other single-stranded RNAs and single-stranded RNAs to single-stranded DNAs; and T4 DNA ligase, available from many commercial sources, is capable of catalyzing both inter- and intramolecular ligation of double-stranded DNAs.

In some embodiments of preparing a target nucleic acid for sequencing, e.g., in a high-throughput sequencing system, double-stranded nucleic acid adapters can be ligated to the ends of double-stranded DNA fragments that comprise the sequence of a target nucleic acid (see, e.g., FIGS. 7A and 7B). In other embodiments, primers comprising adapter sequences at their 5′ ends can be hybridized to a denatured double-stranded DNA fragment of interest. The primers can then be extended, e.g., with a DNA polymerase to produce double-stranded fragments that comprise adapter sequences at each end. DNA polymerases that are typically used to extend primers include, e.g., any of the Taq polymerases, exonuclease deficient Taq polymerases, E. coli DNA Polymerase 1, Klenow fragment, reverse transcriptases, Φ29-related polymerases including wild type Φ29 polymerase and derivatives of such polymerases such as exonuclease deficient forms, T7 DNA Polymerase, T5 DNA Polymerase, T3 DNA polymerase, Pfu DNA polymerase, Vent DNA polymerase, Bst DNA polymerase, etc. Most of the aforementioned DNA polymerases are commercially available from, e.g., New England Biolabs, Roche, Sigma-Aldrich, and others. 9° Nₘ™ DNA polymerase, a thermophilic DNA polymerase that has been genetically engineered to have a decreased 3′→5′ proofreading exonuclease activity, can be of particular use in the methods described herein.

In preferred embodiments of producing a capture probe, a tag can be attached to the 5′ end of a capture probe during a reverse transcription reaction (see, e.g., FIG. 3 and corresponding description.) in which cDNAs comprising 5′ tags are synthesized from RNAs, e.g., RNAs that were transcribed from nucleic acids tethered to a solid support. In general, primers comprising 5′ tags can be annealed to the 3′ ends of the RNAs using, e.g., methods schematically depicted in FIG. 3 (see corresponding description). The primers are extended with a reverse transcriptase to produce the cDNAs comprising a covalently-bound 5′ tags. Further details regarding the synthesis of cDNAs from RNAs, e.g., mRNAs, are elaborated above.

Hybridization

Nucleic acids, e.g., capture probes and target nucleic acids, hybridize due to a variety of well-characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, New York), as well as in Current Protocols in Molecular Biology, Ausubel et al., eds., Current Protocols, a joint venture between Greene Publishing Associates, Inc. and John Wiley & Sons, Inc., (supplemented through 2004) (“Ausubel”); Hames and Higgins (1995) Gene Probes 1 IRL Press at Oxford University Press, Oxford, England, (Hames and Higgins 1) and Hames and Higgins (1995) Gene Probes 2 IRL Press at Oxford University Press, Oxford, England (Hames and Higgins 2).

In general, the stringency of the conditions under which, e.g., capture probes and target nucleic acids, are hybridized, e.g., in the methods described herein, are experimentally determined. An extensive guide to the hybridization of nucleic acids is found in Tijssen (1993), supra. and in Hames and Higgins, 1 and 2. For example, in determining stringent hybridization and wash conditions, the hybridization and wash conditions are gradually increased (e.g., by increasing temperature, decreasing salt concentration, increasing detergent concentration and/or increasing the concentration of organic solvents such as formalin in the hybridization or wash), until a selected set of criteria are met.

### Kits

Kits are also a feature of the invention. The present invention provides kits that incorporate the compositions of the invention, optionally with additional useful reagents such as one or more enzymes that are used in the methods, e.g., an RNA polymerase, a DNA polymerase, a reverse transcriptase, etc., that can be unpackaged in a fashion to enable their use. Depending upon the desired application, the kits of the invention optionally include additional reagents, such as a control target nucleic acids, buffer solutions and/or salt solutions, including, e.g., divalent metal ions, i.e., Mg⁺⁺, Mn⁺⁺ and/or Fe⁺⁺, nucleic acid adapter tags, e.g., to prepare captured nucleic acids for sequencing, etc. Such kits also typically include a container to hold the kit components, instructions for use of the compositions, and other reagents in accordance with the desired application methods, e.g., identifying transcription start sites, identifying exon-exon junctions, and the like.

### EXAMPLES

The following examples are offered to illustrate, but not to limit the claimed invention.

### Example 1

### Using a Capture Probe Comprising a Selector Subsequence Complementary to a Subsequence in a Luciferase mRNA

Single-Stranded Nucleic Acids from Which RNAs can be Synthesized.

An example of a single-stranded nucleic acid from which RNAs can be synthesized, e.g., in preparation to produce a nucleic acid capture probe is shown below: 5′ TGCAGGGCGGACCGATCACATGAAGCAGCACGACTTCATTGCCTATAGTGAGTCGTATTA 3′ (SEQ ID NO: 4). The constant regions are depicted in bold, promoter region is underlined, and the variable selector subsequence is in italic font.

Transcription of RNAs on Solid Surface

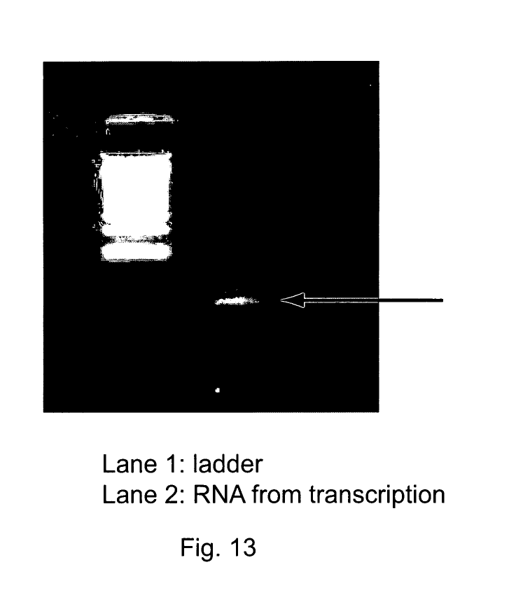

A microarray containing 200,000 unique clusters of DNA oligonucleotides immobilized on a glass surface was purchased from Roche Nimblegen (Madison, Wis.) and hybridized overnight to a population of free oligonucleotides comprising a sequence complementary to the T7 promoter region of the immobilized nucleic acids (5′ TAATACGACTCACTATAGG 3′ (SEQ ID NO: 5)). The hybridization was performed using a final oligo concentration of 100 μM in 30 μl of buffer containing 100 m potassium acetate and 30 mM HEPES. The hybridization was performed using a lifter slip (Thermo Fisher, Portsmouth, N.H.) in an oven at 45° C. overnight to permit the formation of double-stranded promoter regions that can facilitate the transcription of the immobilized nucleic acids by an RNA polymerase. Excess free oligos were washed off the microarray, with 3 room temperature washes of nuclease free water, and 30μl transcription reactions were performed using the T7 MEGAshortscript™ High Yield Transcription Kit (Ambion) at 37° C. for approximately 18 hours. The solution was the collected and the array was washed two times with 10 mM Tris, pH=7.5. Each wash was collected and the resulting RNA was precipitated using ⅓ volume sodium acetate and 2.5 volumes 95% ethanol at −80° C. for 20 minutes. Following the precipitation, RNA was resuspended in 10 μl nuclease free water and contained 1.98 μg of RNA as determined by a Nanodrop™ spectrophotometer (Thermo Fisher). A 2% agarose gel was used to estimate the size and quality of the RNA product (see FIG. 13).

Generating Capture Probe from Transcribed RNA

Capture probes comprising a selector subsequence complementary to a target subsequence in a luciferase mRNA were synthesized as follows. 10 picomoles of a luciferase test oligo (5′ GACTTGTGCAGGGCGGACTATGAAGAGATACGC CCTGCATTGCCCTCTCCCTATAGTGAGTCGTATTAG 3′ (SEQ ID NO: 6)) was hybridized with 10 picomoles of an oligo complementary to the T7 promoter region (5′ TAATACGAC TCACTATAGG 3′ (SEQ ID NO: 7)) in a 20 μl reaction in 1 mM Tris, pH7.5, 0.1M NaCl by heating the sample to 95° C. for 5 minutes followed by 60° C. for 20 minutes, 50° C. for 20 minutes and 37° C. for 20 minutes. 3 picomoles of partially double stranded DNA was used for transcription with the T7 MEGAshortscript™ Kit (Ambion) for 14 hours, following the manufacturers directions. Samples were DNAse treated for 10 minutes and the resulting RNA was precipitated by adding ⅓ volume ammonium acetate and 2.5 volumes of 95% ethanol. Following resuspension in nuclease free water, quantity was determined to be 20 μg using a Nanodrop™ spectrophotometer and quality was assessed by running a sample on a 2% agarose gel.

500 ng of transcribed RNA and 1μl of 50 μM oligo (5′ biotin-GACTTGTGC AGGGCGGA 3′ (SEQ ID NO: 8)) were added to a reverse transcription reaction using the Superscript™ III First-Strand cDNA Synthesis Kit (Invitrogen) according to the manufacturers instructions. The quality of cDNA was assessed by running a sample on a 2% agarose gel.

Oligonucleotide primers comprising a sequence complementary that at the 3′ end of the cDNAs (5′ GGGAGAGGGCAATG 3′ (SEQ ID NO: 9)) were then annealed to the cDNAs and extended with Taq DNA polymerase (Promega) to produce double-stranded cDNAs in a 150 μl reaction consisting of 1× ThermoPol Buffer (NEB), 66 μM dNTPs, 200 μM oligo and 10 U Vent DNA polymerase (NEB). The reaction was heated to 95° C. for 2 min, then incubated at 50° C. for 1 minute followed by 68° C. for 30 seconds. The double stranded cDNAs were bound to 150 μl of streptavidin-coated beads (Myone-C1, Invitrogen) in 1× B&W buffer according to the manufacturers directions. The bound cDNAs were then digested with 2 U BsrD1 in NEB buffer 2 plus BSA at 65° C. overnight to eliminate additional nucleotide bases that were added to the 5′ ends of the RNAs during transcription, and which were accordingly included at the 3′ ends of the cDNAs that were reverse transcribed from the RNAs. Because the selector subsequence is encoded at the unbiotinylated ends of the double-stranded cDNAs, removal of the nucleotides was performed to more efficient hybridization of the capture probes to the target nucleic acid of interest. Following BsrD1digestion, the unbiotinylated strands of each double-stranded cDNA were digested with lambda nuclease (NEB) in 1× lambda nuclease buffer for 10 minutes at 37° C. followed by 10 minutes at 75° C. The beads were then washed 1× in nuclease buffer (NEB) and 2 times in 1× ThermoPol buffer (NEB). Digestion efficiency was assessed by removing oligos from beads by heating to 95° C. and removing the DNA that eluted from the beads. This DNA was then run on a 12% acrylamide gel to determine how much product had been digested

### Capture of Target DNA

HEK 293T cells were grown in IMDM at 37° C. in 5% CO2. Cells were transfected with a plasmid containing a CMV promoter driving firefly luciferase using Lipofectamine™ 2000 according to the manufacturer's directions (Invitrogen). RNA was extracted from the cells 2 days later using the RNA-Bee kit (Amsbio) according to the manufacturer's directions. RNA quality was assessed by agarose gel and quantity was determined with a Nanodrop™ spectrophotometer. 5 μg of total RNA was made into cDNA using the Superscript™ III First-Strand Synthesis System with oligo dT primers according to manufacturer's protocol (Invitrogen)

100 ng of the total cDNA (prepared from RNA harvested from HEK 293T cells expressing luciferase, as described above) was added to 250 ng bead bound capture probes. A single round of PCR was performed in a PCR reaction containing: ThermoPol PCR Buffer (NEB), 0.5 mM dNTPs, 100 ng cDNA, 2 units Vent DNA polymerase (NEB) with one cycle of 95° C. for 2 minutes, 52° C. for 1 minute, 68° C. for 50 seconds. DNA was then heat denatured at 95° C. for 2 minutes and the beads were washed 2 times in 1× ThermoPol buffer leaving single stranded DNA bound to the beads. The DNA on the beads was made double stranded by performing another single round of PCR using 2.5 mM oligo with a known adapter sequence (5′ AATGATACGGCGACCACCGANNNNNNNN 3′ (SEQ ID NO: 10)) at the 5′ end and random octomer at the 3′ end. The beads were washed 1 time in 1 × ThermoPol buffer and 2 times in 1× NEB buffer 2. Double-stranded DNA was removed from the beads via EciI digestion in NEB buffer 2 plus BSA at 37° C. overnight. The digested double stranded DNA was transferred to a new tube where it was phenol/chloroform extracted and EtOH precipitated. 2 μM of A second adapter (a duplex of 5′ TCGTATGCCGTCTTCTG CTTG 3′ (SEQ ID NO: 11) and 5′ CAAGCAGAAGACGGCATACGANN 3′ (SEQ ID NO: 12)) was then ligated on to the resulting DNA with 1 μl High concentration T4 DNA ligase (NEB) in 1× DNA Ligase buffer with 25% PEG 4000 for 10 minutes at room temperature.

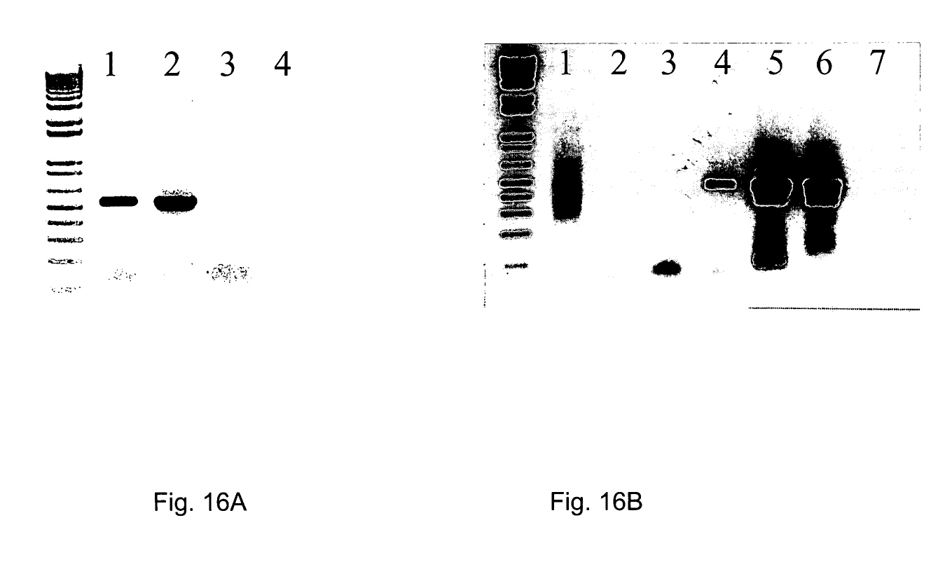

FIG. 16 shows the end result of an experiment in which a capture probe comprising a selector subsequence complementary to the luciferace gene was used. PCR was performed on the captured DNA from either the DNA that had been EciI digested and taken off of the beads (FIG. 16A Lane 1), or the DNA left on the beads (FIG. 16A Lane 2) using luciferase forward and reverse primers (5′ GAACAATTGCTT TTACAGATG 3′ (SEQ ID NO: 13) and 5′ CATTAAAACCGGGAGGTAGA 3′ (SEQ ID NO: 14)). As a negative control, a PCR was performed to detect a gene that would not have been captured using the capture probe described above (primers: 5′ CGTACTAGTATGGAGCAGAAGCTGATCTCAGAGGAGGACCTGATGGATGTATT CATGAAAGG 3′ (SEQ ID NO: 15) and 5′ TCTTAGGCTTCAGGTTC 3′ (SEQ ID NO: 16)). The results of this reaction were run in FIG. 16A Lane 3. A PCR reaction to which no DNA was added was also performed with the aforementioned luciferase primers and was run in FIG. 16A Lane 4. All PCR reactions were 20 μl reactions with 1× PCR Buffer, 2.5 mM MgCl2, 0.5 mM dNTPs, 2 μl template DNA and 5 U Taq with the following reaction times: 95° C. 2 minutes followed by 35 cycles of 95° C. 30 seconds, 53° C. 30 seconds, 72° C. 30 seconds. These results in FIG. 16A indicate the luciferase was enriched in the sample by the capture process, whereas another gene was not.

A second set of PCR reactions is depicted in FIG. 16B. The results of PCR of captured DNA using primers for the 5′ tag (5′ TCGTATGCCGTCTTCTGCTIG 3′ (SEQ ID NO: 17)) and the luciferase reverse primer (5′ CATTAAAACCGGGAGGTAGA 3′ (SEQ ID NO: 18)) is shown in FIG. 16B Lane 1. The smear is the expected result as the length of the captured DNA can vary. A negative control PCR of captured DNA using 5′ tag primer and a primer that should not bind firefly luciferase (5′ AGGTTCTAGAGCTCGAAGCGGCCGCTCT 3′ (SEQ ID NO: 19)) was run is FIG. 16B Lane 2. A PCR performed with a luciferase forward primer (5′ GAACAATTG CTITTACAGATG 3′ (SEQ ID NO: 20)) and a primer that hybridizes to the 3′ tag (5′ AATGATACGGCGACCACCGA 3′ (SEQ ID NO: 21)) was run in FIG. 16B Lane 3. This reaction was expected to give a smear, but the 3′ tag sequence did not work well for PCR in this instance. A PCR reaction performed with the luciferase forward and reverse primers was run in FIG. 16B Lane 4, and the results indicate that the luciferase was captured. A PCR reaction using luciferase primers and the capture beads as template was run in FIG. 16B Lane 5. The results in Lane 5 show that some of the captured DNA was not removed from the beads. A positive control PCR using luciferase primers and input cDNA as a template was run in FIG. 16B Lane 6. FIG. 16B Lane 7 is a negative control PCR with luciferase primers but no template. All PCRs were carried out in 20 μl reactions with 1×PCR buffer, 2.5 mM MgCl2, 0.5 mM dNTPs 0.5 mM primer, 2 μl template and 2.5 U Taq (Promega).

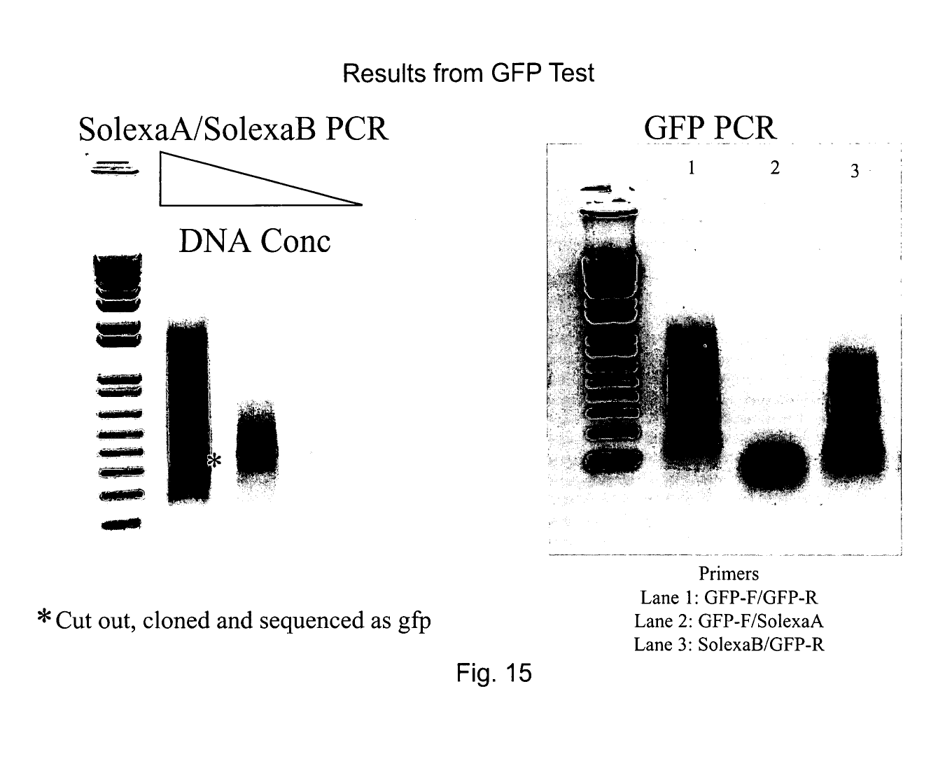

Experiments similar to those described above were performed to obtain the results depicted in FIG. 15, wherein the gene encoding GFP, rather than luciferase, was isolated using the methods and a capture probe of the invention.

### Example 2

### Producing Nucleic Acid Capture Probes from Biotynilated Oligos Attached to Beads

The array used in Example 2 is the same as that described in Example 1. To cleave oligos from the microarray, 35 μl of 28-30% NH₄OH (Sigma catalog no. 221228-25 ml-A) is added to the array, which is then covered with a lifterslip. The array is then incubated at room temperature for two hours. Following the incubation, the liquid is removed from the array and placed in a 1.5 ml microfuge tube. The slide (e.g., the array) and the coverslip are then rinsed twice with 50 μl NH₄OH, which volumes of NH₄OH are then collected and also added to 1.5 ml tube. Water is added to the microfuge tube until the volume of liquid in the tube is about 1.8 ml. The liquid is then transferred to a pre-rinsed YM-3 Centricon tube and spun in a microfuge at 6,500×g for about 2 hours at room temperature (e.g., about 25° C.). Following the first centrifugation, 1 ml of water is added to the Centricon tube, and the tube is spun a second time at a speed of 6,500×g for about 1 hour at room temperature. The Centricon tube is then inverted and spun at a speed of 800×g for about 2 minutes to collect any remaining liquid in the collection tube.

Biotin Tagging and Amplifying Oligos

The following reagents are combined in a PCR reaction tube in a final volume of 50 microliters (i.e., in preparation to amplify and biotinylate the oligos prepared from the step described above):
X μl Template oligos (as described in Example 1)
5 μl NEB Thermopol Buffer 1 μl mM dNTPs
4 μl biotinylated 5′ oligo 2.5 pmol/μl
   (5′ biotin ttgatGATGCATCTGAGCATCTGATgtttaaacTcat GCTGAAG 3′ (SEQ ID NO: 22))
4 μl 3′ oligo 2.5 pmol/μl
   (5′ TAATACGACTCACTATAGGgagataggCAATG 3′(SEQ ID NO: 23))
1 μl Taq polymerase
   The reaction tube is then placed in a thermocycler, which is set to the following program:
95° C. for 2 minutes
95° C. for 45 seconds
53° C. for 1.5 minutes
72° C. for 20 seconds
Goto step 2 24×
4° C. until reactions are retrieved from thermocycler
   The amplified oligos are then captured onto Dynabeads® MyOne™ Streptavidin C1beads (Invitrogen) according to manufacturer's instructions.

Transcribing RNAs from Oligos Immobilized on Beads

RNAs are then transcribed from the bead-immobilized oligos using the Ambion MEGAshortscnpt™ T7 kit following the manufacturers directions. All reagents are RNAse free. The following reagents are mixed and added to the dry beads prepared in the previous step described above:
10 μl water
8 μl total nucleotide (previously mixed A,T,C,G; each of which are 75 mM)
2 μl T7 enzyme mix (which comprises an RNAse inhibitor)
   The reaction mix is incubated at 37° C. for about 6 hours, after which the beads are pelleted using a magnetic stand. The supernatant is transferred to a 1.5 ml microfuge tube. The beads are then washed in 50 μl RNAse-free water, pelleted as described, and the supernatant from this step is also transferred to same 1.5 ml tube as the supernatant from the previous step. The following reagents are added to the 1.5 ml tube:
95 μl RNAse-free water
15 μl NH₄O-Acetate (5M)
1 μl LPA (linear polyacrylamide)
150 μl Isopropanol
   This reaction mix is then incubated at −80° C. for 15 minutes or, alternately, at −20° C. overnight to precipitate the RNA produced during the transcription reaction. Following the incubation, the tube is then spun in a microfuge at maximum speed for 15 minutes at 4° C. The supernatant is removed, and the pellet is washed twice with 70% ethanol. After the pellet has been washed and dried, it is resuspended in 12 μl of RNAse-free water. A sample of RNA can be removed to determine its concentration according to its optical density (OD) at 260 nm. Alternately, the RNA can be purified using a Qiagen RNA extraction column according to manufacturer's instructions.

Reverse Transcription

Typically, 200 ng of RNA produced as described above is then used in a reverse transcription reaction, e.g., in order to produce nucleic acid capture probes. The reaction is performed using an Invitrogen SuperScript® III kit according to manufacturer's instructions. To reverse transcribe the RNA, the following reagents are added to a 50 μl RNAse free microfuge tube:
1 μl of 5 μM biotin RT oligo
   (5′biotin- gtgcattgaattcgaccactaaaggAACTCATGCTGAAG 3′ (SEQ ID NO: 24))
1 μl 10 mM dNTPs
Add water to 10 μl
   The reaction mix is then heated at 65° C. for 5 minutes and then placed on ice for 2 minutes to permit the RT oligos to anneal to the RNAs. The following reaction mix is prepared, and the reagents, provided by the Invitrogen SuperScript® III kit, are added to the mix in the order in which they are listed):
2 μl 10× RT buffer
4 μl 25 mM MgCl₂
2 μl 0.1M DTT
1 μl RNAseOUT
1 μl SuperScript® III (reverse transcriptase)
   10μl of the above mix is added to the tube on ice that contains the RNA, and the reverse transcriptase reaction is then incubated at 50° C. for at least 2 hours. The reaction can optionally proceed overnight. To stop reverse transcription, the reaction tube is heated to 85° C. for 5 minutes, after which it is placed on ice. The tube is spun briefly in a microfuge to collect condensation. To digest the RNA, 1 μl (2 units) RNAseH packaged with the SuperScript® III kit the is added to the tube, which is then incubated at 37° C. for about 30 minutes.

Second Strand Synthesis

The following reagents are added to the tube from the previous step:
73 μl water
30 μl 5× Taq polymerase buffer (Promega)
15 μl 25 mM MgCl₂
2 μl 10 mM dNTPs
8 μl 10 μM second strand oligo
   (5′ tgcgaattcGGGAGAGGGCAATG 3′ (SEQ ID NO: 25))
2 μl (5 u/ul) Taq (Promega)
   The reaction is heat to 95° C. for 2 minutes, cooled to 50° C. for 1 minute to permit oligo hybridization, and then heated to 72° C. for 2 minutes to permit the synthesis of a complementary DNA strand. The double-stranded DNA is then purified using a Qiagen spin column according to the manufacturer's instructions for PCR reaction purification. The DNA is then eluted from the Qiagen spin column with 50 μl of water. 10 μl NEB buffer 2, 10 μl 10× BSA, 2 μl Bsrd1, and water are added to the purified cDNA such that the final reaction volume is 100 μl. The restriction digest is then incubated at 65° C. for 1 hour.

Removal of Second Strand

The biotinylated oligos are captured Dynabeads® MyOne™ Streptavidin C1 beads (Invitrogen) according to manufacturer's instructions, and the supernatant is removed. The non-biotinylated DNA strand is removed from the bead-captured strand by NaOH incubation. The beads are then left with single stranded capture probes for downstream applications, e.g., any one or more of the applications described above.

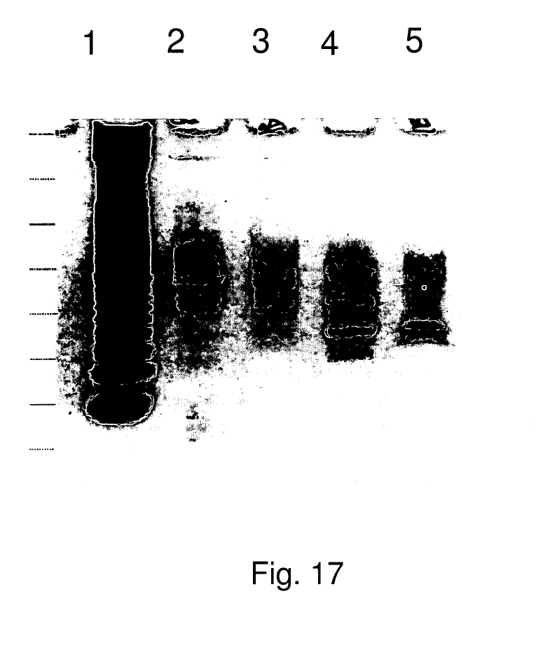

For example, FIG. 17 shows a gel on which samples from each step above, e.g., each step in the preparation of a capture probe, were run. 1 kb+ ladder was run in lane 1. Biotinylated double-stranded capture probe precursors were run in lane 2. DNA that did not bind streptavidin beads was run in lane 3. Biotinylated double-stranded capture probe precursors digested with BsrD1 were run in lane 4. Digested DNA that did not bind streptavidin beads was run in lane 5. These results confirm that the DNA capture probes being produced are digested as expected with the appropriate restriction endonuclease and attach as expected to streptavidin beads.

Capture of cDNA Fragments

The following reagents are added to 300 ng of bead-bound single-stranded capture probes:
about 800 ng cDNA (The cDNA is produced from 800ng RNA that has been reverse transcribed using the Invitrogen SuperScript® III kit according to the manufacturer's protocol. Typically, the volume is approximately 45 μl)
11 μl 5× hybridization buffer (40 mM HEPES pH=7.2; 500 mM NaCl; 1 mM EDTA)
   The capture probes and the cDNA are then incubated on a rocking platform in hybridization buffer at 55° C. overnight.

Primer Extension

The bead-bound capture probes, e.g., to which “captured” cDNAs have hybridized, are then pelleted using a magnetic stand, and the supernatant is removed. The following premixed reaction mix is then added to the pelleted beads:
20 μl 5× PCR buffer (Promega)
9 μl 25 mM MgCl₂
1 μl 10 mM dNTP
69 μl water
1 μl Taq
   The beads are resuspended in the reaction mix and incubated at 65° C. for 10 minutes. Following the incubation, the beads are washed twice with water. The beads are then incubated in 1.5 M NaOH for 10 minutes at room temperature to denature double-stranded nucleic acids. The beads are then washed twice in 2× Invitrogen Bind and Wash (B&W) buffer (10 mM Tris pH=7.5, 1 mM EDTA, 0.2M NaCl).

The beads can now be used in PCR reactions with Solexa primers to prepare the captured nucleic acids for sequencing, e.g., in a high-throughput sequencing system. For example, a 50 μl PCR reaction is prepared:
1 μl 10mM Solexa primer 5′ AATGATACGGCGACCACCGA 3′ (SEQ ID NO: 26)
1 μl 10 mM Solexa primer 5′ CAAGCAGAAGACGGCATACGA 3′ (SEQ ID NO: 27)
1 μl 10 mM dNTP
10 μl 5× Phusion reaction buffer
0.5 μl Phusion enzyme (2 unit/μl)
   Phusion polymerase is available from Finnzymes. Water is added to the reaction until the volume reaches 50 ul. The reaction is then placed in a thermocycler set to run on the following program:
98° 30 sec
98° 5 sec
63° 20 sec
72° 10 sec
Go to step 2 9 times
4° until the reaction is retrieved from the thermocycler.
   The PCR reaction is then purified using a Qiagen spin column according the manufacturer's instructions, and the DNA from the PCR reaction can be quantified using Syber Green (available from Sigma Aldrich) according to the manufacturer's instructions.

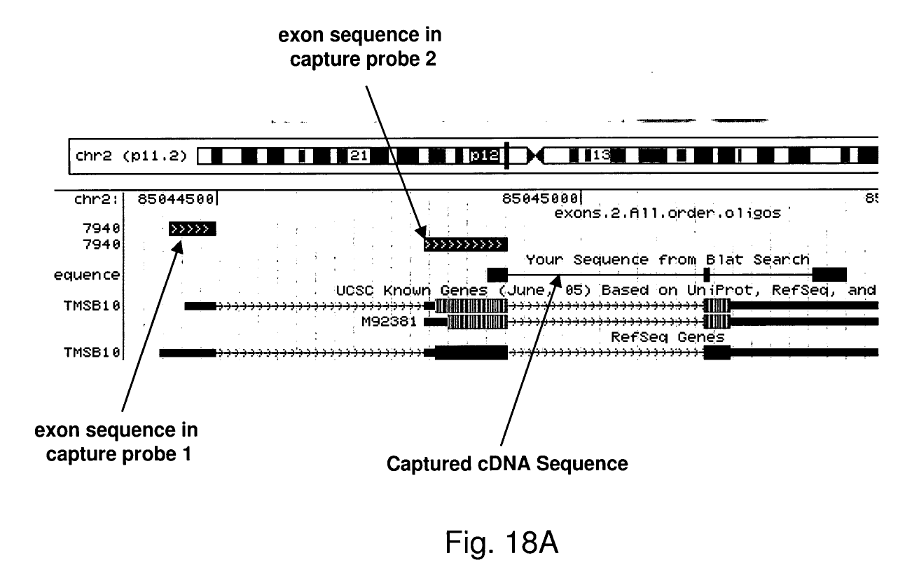

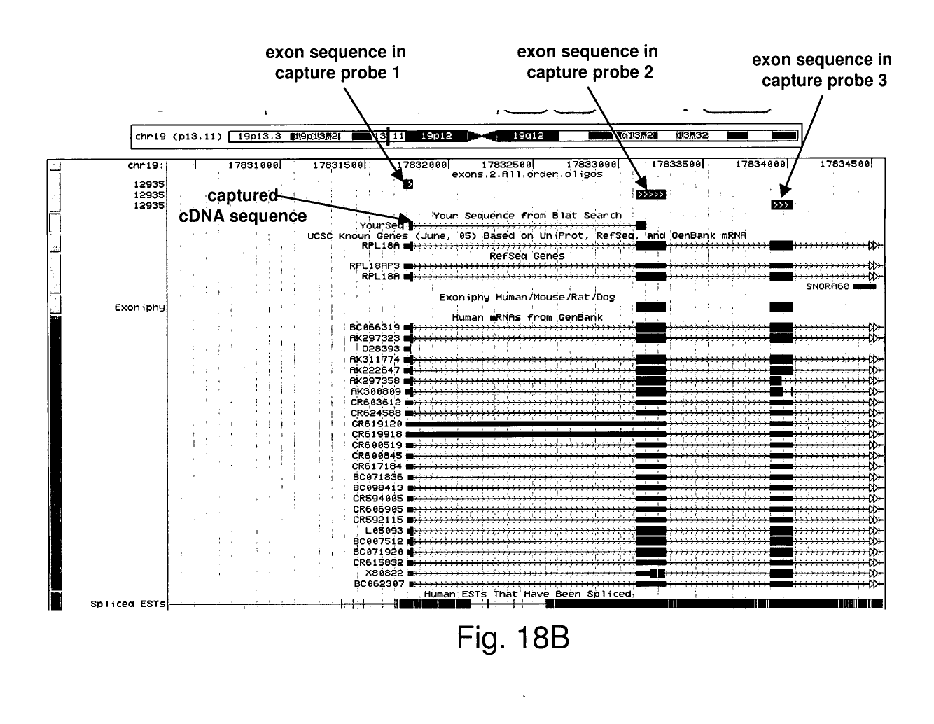

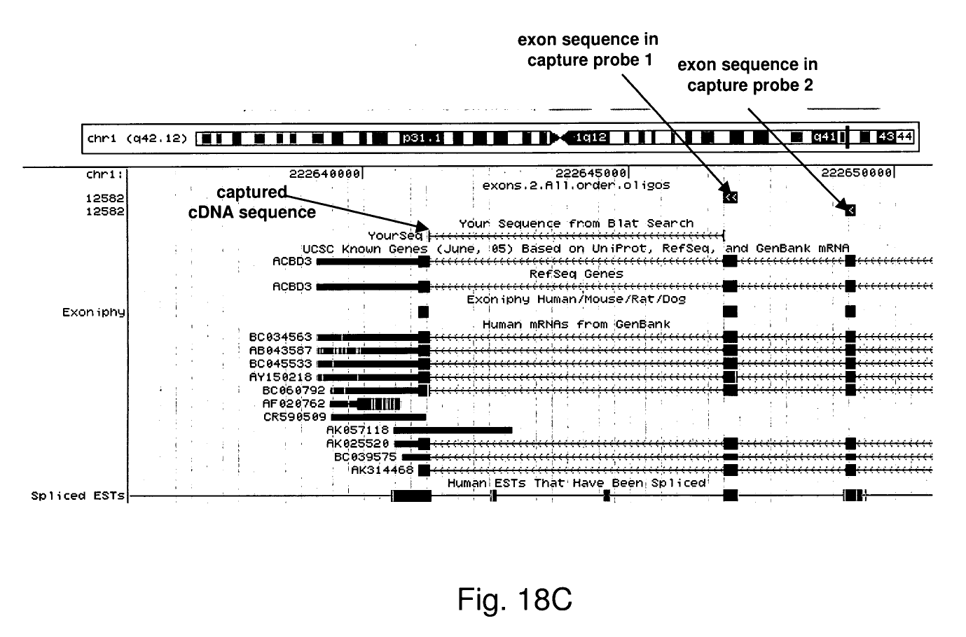

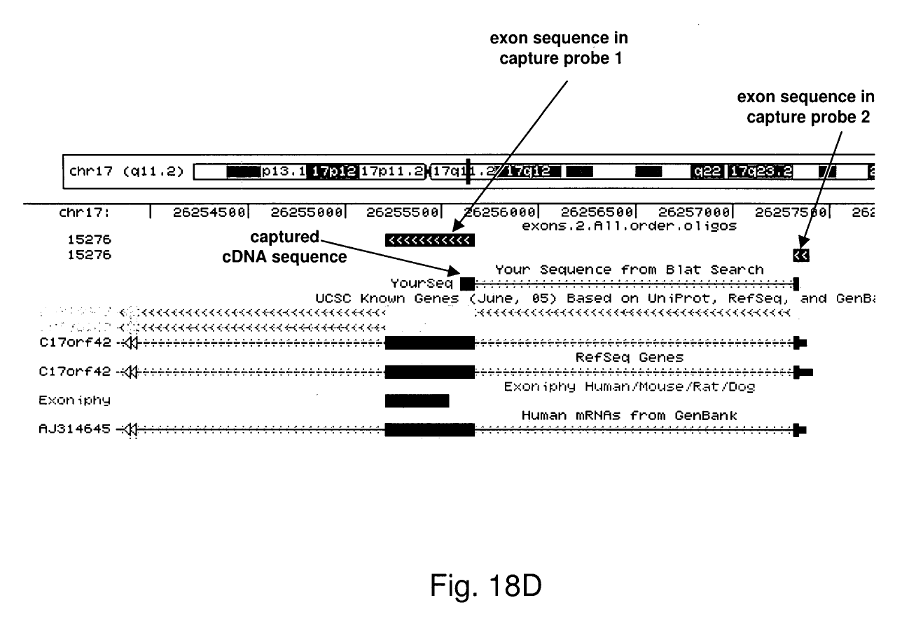

FIG. 18A-D shows data for a capture experiment that was performed according to the protocol described above. The exon sequences included in the capture probes used in each experiment, as well as the cDNA sequence that was captured by the probes, are indicated in each panel A-D. Each exon sequence in FIG. 18A-18D indicates a separate capture probe.

It is understood that the examples and embodiments described herein are for illustrative purposes only and that various modifications or changes in light thereof will be suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A composition, comprising:
a solid support; and,
at least one nucleic acid, wherein a 5′ end of the nucleic acid is tethered to the solid support, and wherein a 3′ end region of the nucleic acid comprises at least one strand of a promoter sequence recognized by an RNA polymerase, and wherein the nucleic acid is capable of being transcribed by the RNA polymerase from the promoter towards the 5′ end when the promoter sequence is sufficiently double stranded for recognition by the RNA polymerase.

2. The composition of claim 1, wherein the solid support comprises a polymer, a ceramic, glass, a metal, a metalloid, or a magnetic material.

3. The composition of claim 1, wherein the solid support comprises a planar substrate, a bead, a slide, a microscope slide, or a micro-well plate.

4. The composition of claim 1, wherein the nucleic acid comprises a selector subsequence of interest downstream of the promoter sequence, wherein the selector subsequence can be transcribed by the RNA polymerase.

5. The composition of claim 4, wherein the selector subsequence comprises or encodes an exon, an intron, an exon-exon boundary, a 3′UTR/polyA site, a transcription start site, an shRNA sequence, or a subsequence of an miRNA.

6. The composition of claim 4, wherein the nucleic acid comprises a constant region downstream of the selector subsequence.

7. The composition of claim 6, wherein the constant region comprises or encodes at least one strand of a unique restriction endonuclease recognition site.

8. The composition of claim 1, wherein the promoter sequence is selected from the group consisting of: a T7 promoter, a T3 promoter, and an SP6 promoter.

9. The composition of claim 1, comprising a primer, which primer hybridizes to the promoter sequence, permitting the RNA polymerase to transcribe the nucleic acid downstream of the promoter.

10. The composition of claim 9, wherein the RNA polymerase is selected from the group consisting of: a T4 RNA polymerase, T7 RNA polymerase, a T3 RNA polymerase, and an SP6 RNA polymerase.

11. The composition of claim 1, wherein the composition comprises an array of nucleic acids on the solid support, the array comprising a plurality of copies of each of a plurality of nucleic acid sequence types.

12. The composition of claim 11, wherein the nucleic acid sequence types comprise a plurality of selector subsequences, each comprising an exon, an intron, an exon-exon boundary, a 3′UTR/polyA site, a transcription start site, an shRNA sequence, or a subsequence of an miRNA.

13. A system comprising the composition of claim 1, which system additionally comprises a production module that produces transcripts of the nucleic acid.

14. The system of claim 13, further comprising a processing module that copies or transcribes the transcript and a sequencing module that sequences products of the processing module.

15. A method of producing an RNA, the method comprising:
providing a solid support to which at least one nucleic acid is tethered at a 5′ end of the nucleic acid, and wherein a 3′ end region of the nucleic acid comprises at least one strand of a promoter sequence recognized by an RNA polymerase;
annealing a primer to the promoter sequence to provide the promoter recognized by the RNA polymerase; and,
transcribing the nucleic acid with the RNA polymerase, wherein the polymerase travels along the nucleic acid toward the 5′ end during transcription, thereby producing the RNA.

16. The method of claim 15, comprising chemically or enzymatically coupling the nucleic acid to the solid support.

17. The method of claim 15, further comprising producing a cDNA from the RNA.

18. The method of claim 17, further comprising sequencing at least a portion of the cDNA or a complementary sequence thereof.

19. A method of synthesizing a tagged single-stranded nucleic acid capture probe, the method comprising:
providing a solid support to which at least one nucleic acid has been tethered at a 5′end, wherein the nucleic acid comprises a selector subsequence of interest and at least one strand of a promoter sequence recognized by an RNA polymerase upstream of the selector subsequence;
transcribing the nucleic acid with the RNA polymerase to produce an RNA;
reverse transcribing the RNA with a reverse transcriptase to produce a tagged single-stranded cDNA; and,
removing at least one nucleotide from the 3′ end of the tagged single-stranded cDNA, thereby producing the tagged single-stranded capture nucleic acid.

20. The method of claim 19, wherein the promoter is double stranded, wherein the promoter comprises a primer annealed to the nucleic acid.

21. The method of claim 19, wherein reverse transcribing the RNA comprises:
annealing a tagged primer to a 3′ end of the RNA and extending the tagged primer with a reverse transcriptase to form an RNA:DNA duplex comprising a cDNA strand with a tagged 5′ end; and,
separating an RNA strand from the tagged cDNA strand.

22. The method of claim 21, wherein annealing a tagged primer to the 3′ end of the RNA comprises annealing a primer that is complementary to a sequence at the 3′ end of the RNA, wherein a 5′ end of the primer comprises one or more phosphorylated nucleotide, phosphorothioated nucleotide, biotinylated nucleotide, digoxigenin-labeled nucleotide, methylated nucleotide, uracil, sequence capable of forming hairpin secondary structure, oligonucleotide hybridization site, restriction endonuclease recognition site, or cis regulatory sequence.

23. The method of claim 21, wherein annealing the tagged primer to the 3′ end of the RNA comprises:
adding a polyA tail to the 3′ end of the RNA; and,
annealing a polyT primer to the polyA tail, wherein a 5′ end of the polyT primer comprises one or more phosphorylated nucleotide, phosphorothioated nucleotide, biotinylated nucleotide, digoxigenin-labeled nucleotide, methylated nucleotide, uracil, sequence capable of forming hairpin secondary structure, oligonucleotide hybridization site, restriction site, or cis regulatory sequence.

24. The method of claim 23, wherein the polyA tail is added to the 3′ end of the RNA by enzymatic addition of adenosine residues by a polyA polymerase, a terminal transferase, or an RNA ligase.

25. The method of claim 21, wherein separating an RNA strand from the tagged cDNA strand comprises denaturing the RNA-DNA duplex.

26. The method of claim 21, wherein separating an RNA strand from the tagged cDNA strand comprises digesting the RNA strand of the RNA-DNA duplex with RNAse H.

27. The method of claim 19, wherein removing at least one nucleotide from the 3′ end of the tagged single-stranded DNA comprises digesting the tagged single-stranded DNA with an enzyme that has a 3′ to 5′ exonuclease activity.

28. The method of claim 19, comprising sequencing at least a portion of the tagged single-stranded capture nucleic acid, or a complementary sequence thereof.

29. A method of synthesizing a single-stranded nucleic acid capture probe, the method comprising:
providing a solid support to which at least one nucleic acid has been tethered at a 5′end, wherein the nucleic acid comprises a selector subsequence of interest and at least one strand of a promoter sequence recognized by an RNA polymerase upstream of the selector subsequence;
transcribing the nucleic acid with the RNA polymerase to produce an RNA;
reverse transcribing the RNA with a reverse transcriptase to produce a double-stranded cDNA with one tagged end;
removing at least one nucleotide base pair from an untagged end of the double-stranded cDNA; and,
separating the strands of the double-stranded cDNA from one another, thereby producing the tagged single-stranded capture nucleic acid.

30. The method of claim 29, wherein the promoter is double stranded, wherein the promoter comprises a primer annealed to the nucleic acid

31. The method of claim 29, wherein reverse transcribing the RNA comprises:
annealing a tagged primer to a 3′ end of the RNA;
extending the tagged primer with a reverse transcriptase to form a double-stranded RNA-DNA duplex comprising a cDNA strand with a tagged 5′ end;
separating strands comprising the RNA-DNA duplex to produce an RNA strand and a tagged cDNA strand; and
annealing an untagged primer to a 3′ end of the tagged cDNA strand and extending the untagged primer with a DNA polymerase to produce the double-stranded cDNA that comprises one tagged strand.

32. The method of claim 31, wherein annealing the tagged primer to the 3′ end of the RNA comprises annealing a primer that is complementary to a sequence at the 3′ end of the RNA, wherein a 5′ end of the primer comprises one or more phosphorylated nucleotide, phosphorothioated nucleotide, biotinylated nucleotide, digoxigenin-labeled nucleotide, methylated nucleotide, uracil, sequence capable of forming hairpin secondary structure, oligonucleotide hybridization site, restriction endonuclease recognition site, or cis regulatory sequence.

33. The method of claim 31, wherein annealing the tagged primer to a 3′ end of the RNA comprises:
adding a polyA tail to the 3′ end of the RNA; and,
annealing a polyT primer to the polyA tail, wherein a 5′ end of the polyT primer comprises one or more phosphorylated nucleotide, phosphorothioated nucleotide, biotinylated nucleotide, digoxigenin-labeled nucleotide, methylated nucleotide, uracil, sequence capable of forming hairpin secondary structure, oligonucleotide hybridization site, restriction site, or cis regulatory sequence.

34. The method of claim 34, wherein the polyA tail is added to the 3′ end of the RNA by the enzymatic addition of adenosine residues by a polyA polymerase, a terminal transferase, or an RNA ligase.

35. The method of claim 31, wherein the DNA polymerase is selected from the group consisting of: an E. coli DNA polymerase I, a Taq polymerase, a T7 DNA polymerase, a T3 DNA polymerase, a phi29 DNA polymerase, a Vent DNA polymerase, a Pfu DNA polymerase, a Bst DNA polymerase, and a 9° Nm™ DNA polymerase.

36. The method of claim 29, wherein removing the at least one nucleotide base pair from the untagged end of the double-stranded cDNA comprises digesting the double-stranded cDNA with an endonuclease at a site proximal to the untagged end of the double-stranded cDNA such that the nucleotide base pair is removed from the double-stranded cDNA.

37. The method of claim 29, wherein separating the strands of the double-stranded cDNA comprises denaturing the double-stranded cDNA, thereby producing the tagged, single-stranded capture nucleic acid.

38. The method of claim 29, wherein separating the strands of the double-stranded cDNA comprises digesting an untagged strand with a lambda nuclease, thereby producing the tagged, single-stranded capture nucleic acid.

39. The method of claim 29, comprising sequencing at least a portion of the tagged single-stranded capture nucleic acid, or a complementary sequence thereof.

40. A nucleic acid library, comprising:
one or more arrays, wherein each array comprises a solid support and a plurality of nucleic acids, wherein first ends of the nucleic acids are tethered to the solid support, wherein each of the plurality of nucleic acids comprises a strand of an RNA polymerase promoter sequence and a unique selector subsequence downstream of the promoter sequence, and wherein each of the plurality of nucleic acids can be transcribed to produce an RNA encoding the selector subsequence by annealing a primer to the promoter sequence such that the promoter is recognized by an RNA polymerase, and transcribing the nucleic acid with the RNA polymerase.

41. The nucleic acid library of claim 40, wherein the solid support comprises a polymer, a ceramic, a metal, a metalloid or a magnetic material.

42. The nucleic acid library of claim 40, wherein the solid support comprises a planar substrate, a bead, a slide, a microscope slide, or a micro-well plate.

43. The nucleic acid library of claim 40, wherein the nucleic acids are tethered at 5′ ends of the nucleic acids to the solid support.

44. The nucleic acid library of claim 40, wherein the nucleic acids each comprise or encode a strand of one or more unique restriction endonuclease recognition site.

45. The nucleic acid library of claim 40, wherein the selector subsequences each comprise an exon, an intron, an exon-exon junction, a 3′UTR/polyA site, a transcription start site, an shRNA or a subsequence of an miRNA.

46. The nucleic acid library of claim 40, wherein the nucleic acids each comprise a constant subsequence downstream of the selector subsequence.

47. The nucleic acid library of claim 46, wherein the constant regions each comprise or encode a strand of one or more unique restriction endonuclease recognition site.

48. The nucleic acid library of claim 40, wherein the promoter sequence is selected from the group consisting of: a T7 promoter, a T3 promoter, and an SP6 promoter.

49. A nucleic acid exon library, comprising:
an array of nucleic acids each comprising an upstream exon or exon subsequence and a processing feature subsequence that facilitates interrogation of a target nucleic acid with the exon or exon subsequence to determine the sequence of a downstream exon sequence found in the target nucleic acid.

50. The library of claim 49, wherein the nucleic acids are single stranded.

51. The library of claim 49, wherein the nucleic acids are bound to a solid support.

52. The library of claim 49, wherein the processing feature comprises a promoter facilitating transcription of the nucleic acids of the array.

53. The library of claim 49, wherein the processing feature comprises or encodes a restriction endonuclease recognition site.

54. A method of determining a sequence of an exon-exon junction in a target nucleic acid, the method comprising:
providing an array of nucleic acids, wherein each nucleic acid comprises one exon or exon subsequence;
producing one or more capture probes from the array of nucleic acids, wherein each capture probe comprises or encodes at least a portion of the exon or exon subsequence present in each nucleic acid in the array; and,
sequencing at least a portion of one or more target nucleic acids captured using the one or more capture probes, thereby determining the sequence of the exon-exon junction.

55. The method of claim 54, wherein sequencing one or more target nucleic acid comprises:
providing a population of nucleic acids; and,
hybridizing the one or more capture probes to one or more target nucleic acids in the population, which target nucleic acids comprise a subsequence complementary to the exon subsequence of the probes, to produce at least one target nucleic acid-bound probe;
separating the target nucleic acid-bound probe from unbound nucleic acids;
extending recessed 3′ ends of strands of the target nucleic acid-bound probe with a DNA polymerase to produce a double-stranded fragment;
attaching tags to ends of the double stranded fragments, wherein the tags comprise primer hybridization sites to produce tagged fragments; and,
transferring the tagged fragments to a reaction volume that contains a mixture of sequencing reagents; and,
performing a sequencing reaction.
